(19) United States
(12) Patent Application Publication
(10) Pub. No.: US 2025/0261851 A1
(43) Pub. Date: Aug. 21, 2025

(54) METHOD AND APPARATUS OF MULTI-SPECTRAL RETINAL IMAGING WITH WIDE FIELD OF VIEW

(71) Applicant: AI-SPECTRAL TECHNOLOGY CORP., Kanata (CA)

(72) Inventors: Nick Zeljko Ribaric, Ottawa (CA); Kexing Liu, Nepean (CA); David Tawei Yi, Kanata (CA); Matthieu Aubrey-Horvath, Ottawa (CA); Robert Andrews, Nepean (CA); Ian Powell, Ottawa (CA); Alborz Ehteshami, Ottawa (CA); Andrew John MacPhail, Ottawa (CA)

(21) Appl. No.: 19/054,692

(22) Filed: Feb. 14, 2025

Related U.S. Application Data
(60) Provisional application No. 63/554,491, filed on Feb. 16, 2024.
(60) Provisional application No. 63/658,517, filed on Jun. 11, 2024.

(51) Int. Cl. A61B 3/12; A61B 3/00; A61B 3/10

(52) U.S. Cl. CPC A61B 3/1225; A61B 3/0008; A61B 3/1015

(57) ABSTRACT

Apparatus and method for multi-spectral retinal imaging with wide field of view including multi-spectral fundus auto fluorescence. The present disclosure describes an apparatus having a light source assembly for multi-spectral retinal illumination for wide field of view retinal imaging including fluorescence retinal imaging. The present apparatus and methods can obtain retinal images at a wide range of spectral wavelengths to provide an effective, efficient, and extended retinal disease identification, monitoring and diagnostics.

### CROSS-REFERENCE TO RELATED APPLICATIONS

This application claims priority to U.S. provisional patent application 63/554,491 filed on 16 Feb. 2024 and U.S. provisional patent application 63/658,517 filed on 11 Jun. 2024, all of which are hereby incorporated by reference herein in their entirety.

### FIELD OF THE INVENTION

This disclosure relates to an apparatus and method for multi-spectral retinal imaging with wide field of view including multi-spectral fundus auto fluorescence. The present disclosure describes a light source assembly for retinal illumination for wide field of view retinal imaging. The present apparatus and methods obtain multi spectral images to provide effective, efficient, and extended retinal disease identification, monitoring and diagnostics.

### BACKGROUND

Retinal imaging techniques are used for early detection and diagnosis of ocular pathologies such as age-related macular degeneration (AMD), diabetic retinopathy and glaucoma. Retinal imaging techniques are also widely used to observe changes in retinal structure and to detect biomarkers, such as to observe changes in retinal structure such as hems, serum leakage, micro aneurisms, fluid accumulation, blood deposits, retina atrophy, blood vessels change and tortuosity, atrophy of retinal pigment epithelium (RPE) and choroid etc. In diabetic retinopathy, for example, retinal imaging techniques may be used for early detection and diagnosis through the observation of decreased blood flow in the retina. The effects of hydroxychloroquine, an arthritis drug, can also be observed through the damage that it causes to the retina. For patients, ongoing retinal imaging can be used to observe the eye to track changes to the fundus to determine disease progress and alert health care providers to any changes in eye health.

A number of diseases can cause damage to the retina leading to a loss of eyesight. Retinal imaging, as part of the clinical care and management of eye and systemic diseases, involves collecting images of the fundus, which is the inside and back surface of the eye, for use in the diagnosis of ocular and systemic disease.

Methods of collecting retinal images include, for example, retinal fundus photography, optical coherence tomography, multi spectral ophthalmoscopes and fundus autofluorescence. Retinal fundus photography generally uses conventional cameras to provide a color images of the fundus. With a conventional camera the central field of view of the retina, such as between about 45 to 60 degrees can be captured, such that the optic nerve and macula, which is in the center of the retina at the back of the eye, can be imaged. However, the visual indicators of certain diseases, for example glaucoma, may start from the periphery of the retina rather than at the center near the macula, and the inability to visualize a full retinal field of view can delay diagnosis of some diseases. Optical coherence tomography, another retinal imaging technique, generates a cross-sectional view of the retina from infrared light reflections and can be used to diagnose drusen, wet and dry AMD, among other conditions. Multi-spectral retinal ophthalmoscopes employ light of different wavelengths ranging from green (525 nm) to near infra-red (900 nm) to observe different layers of the retina. The longer wavelengths penetrate further and provide image of deeper layers of the retina. Fundus autofluorescence uses the fluorophores present in retinal tissues to visualize their presence in the retina and to track change in their fluorescence properties that resulting from aging and/or disease. Different wavelengths of light excite different fluorophores, which then fluoresce at various wavelengths. In an example, age-related macular degeneration in patients can be detected by different retinal pigment epithelium changes using near infrared autofluorescence (787 nm). The fluorescent spectrum can also be used to map the topography of endogenous fluorophores like lipofuscin and melanin. Imaging techniques can be limited by a traditional fundus camera with only a 45 degrees field of view whereas more than 120-degree field of view of the retina is needed to monitor and diagnose diseases occurring on the back of the eye.

To obtain improved field of view retinal imaging, wide field fundus cameras can be used. In one example of a wide field fundus camera, U.S. Pat. No. 11,744,460B2 to Yates and Lai describes a wide field fundus camera disclosed to implement multiple illumination beam projectors and to capture multiple retinal images at various viewing angles to mimic wide field retinal examination with an indirect ophthalmoscope. In another example, a system and method for in vivo detection of fluorescence from an eye is described in U.S. Pat. No. 10,314,473B2 to Smith and involves the in vivo detection and quantification of drusen present in the retina via administering an excitation signal to the retina of the eye and detecting an electromagnetic emissions spectrum from the retina in response to the excitation signal.

Retinal tissues exhibit fluorescence emission upon excitation by suitable wavelengths of light. A large variety of these ocular or retinal fluorophores exhibit marked changes in their fluorescence properties with respect to age and pathology. Thus, the biological and functional properties of retinal tissues and their modifications are indicators of aging or disease and can be used as diagnostic tools. There remains a need for an apparatus capable of collecting high quality wide field multi-spectral retinal images and auto fluorescent images that can be used to diagnose ocular and systemic diseases.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide an apparatus and method for multi-spectral retinal imaging with wide field of view including multi-spectral fundus auto fluorescence.

In an aspect there is provided an apparatus for multi-spectral retinal imaging comprising: a light source assembly comprising a plurality of pairs of opposed illumination assemblies, each illumination assembly comprising: a first light source configured to emit a first light beam at a first peak wavelength; a second light source configured to emit a second light beam at a second peak wavelength; a dichroic mirror to receive light from at least one of the first light source and the second light source and provide an illumination light beam; a light shaping lens to receive the illumination light beam from the dichroic mirror; and a directional optical component to direct the illumination light beam at an inclination angle to a detection axis; a filter assembly comprising an illumination light filter to receive the illumination light beam and an imaging light filter to receive an imaging light beam; an objective lens to direct the illumination light beam onto a retina; and an illumination detector on the detection axis to receive projected light from the retina through the imaging light filter, wherein the inclination angle of the illumination light beam from both of the illumination assemblies in each pair of opposed illumination assemblies is coplanar with the detection axis.

In an embodiment, the first light source and the second light source in each illumination assembly have different peak wavelengths.

In another embodiment, the dichroic mirror has a first side that allows light at the first peak wavelength to be transmitted and a second side that allows light at the second peak wavelength to be reflected.

In another embodiment, the first light beam is orthogonal to the second light beam.

In another embodiment, the illumination light filter is an outer polarization ring at a first polarization and the imaging light beam is an inner polarization disc at a second polarization orthogonal to the first polarization.

In another embodiment, the illumination light filter is an outer color filter ring with a first color filter and the imaging light beam is an inner color filter disc of a second color filter.

In another embodiment, the inclination angle between the illumination light beam and the detection axis is between 2 and 8 degrees.

In another embodiment the further comprises a photosensor to measure illumination intensity of the illumination light beam.

In another embodiment, the filter assembly comprises one or more of polarizing glass, film, broadband metal wire grid polarizer, and aluminum MicroWires.

In another embodiment, the first light source and the second light source comprise one or more of a light emitting diode (LED), LED array, fiber-optic light source, hyper-spectrum laser, wideband tunable laser, and super luminescent diode.

In another embodiment, the imaging sensor is a camera, monochromatic digital image sensor, Complementary Metal-Oxide-Semiconductor (CMOS) sensor, or compound semiconductor sensor.

In another embodiment, the first peak wavelength and the second peak wavelength are between 400 nm to 950 nm.

In another embodiment the apparatus further comprises: a filter cassette comprising one or more filter assembly; and an actuator to actuate positioning of the filter cassette relative to the detection axis.

In another embodiment, the light source assembly further comprises a fixation target.

In another aspect there is provided a method for multi-spectral retinal imaging comprising: selecting a plurality of imaging wavelengths for retinal imaging; and acquiring a wide field of view retinal image for each of the selected imaging wavelengths by: aligning a filter assembly along an imaging detection axis, the filter assembly comprising an outer annular illumination light filter and an inner imaging light filter, the illumination light filter and imaging light filter selected for the selected imaging wavelength; simultaneously directing light from a first light source through a first dichroic mirror in a first illumination light path at a first inclination angle to the detection axis through the illumination light filter and directing light from a second light source through a second dichroic mirror in a second illumination light path at a second inclination angle to the detection axis through the illumination light filter to illuminate the retina, the first illumination light path and the second illumination light path coplanar with the detection axis; and receiving imaging light from the retina through the imaging light filter at an imaging sensor along the detection axis to provide a wide field of view retinal image at the selected imaging wavelength.

In an embodiment, the first light source and the second light source are illuminated simultaneously for between 10 and 250 milliseconds.

In another embodiment, the first inclination angle and the second inclination angle to the detection axis is between 2 and 8 degrees.

In another embodiment, the first light source and the second light source have a peak wavelength between 400 nm to 950 nm.

In another embodiment, for each of the plurality of selected imaging wavelengths the first light source and the second light source have the same peak wavelength.

In another embodiment, the illumination light filter and the imaging light filter are cross-polarized or are color filters in different spectral ranges.

Embodiments of the present invention as recited herein may be combined in any combination or permutation.

### BRIEF DESCRIPTION OF THE FIGURES

For a better understanding of the present invention, as well as other aspects and further features thereof, reference is made to the following description which is to be used in conjunction with the accompanying drawings.

FIG. 1 is a perspective view of an apparatus for multi-spectral retinal imaging with a wide field of view.

FIG. 2 is a cross-sectional schematic of a multi-spectral retinal imaging apparatus with a wide field of view.

FIG. 3A illustrates a light source assembly with four illumination assemblies.

FIG. 3B illustrates a light source assembly with eight illumination assemblies.

FIG. 4A is a cross-sectional schematic of a beam path from an illuminated light source in a light source assembly and light shaping objective lens.

FIG. 4B is a cross-sectional schematic of a beam path from an illuminated light source with a fixation target.

FIG. 5A is an schematic of a fixation target with light paths to the eye.

FIG. 5B is an illustration of fixation target as perceived by a patient.

FIG. 6A illustrates an annular polarizer with inner polarization disc and outer polarization ring with a light source assembly for multi-spectral imaging.

FIG. 6B is a cross-sectional view of a polarizer assembly with offset illumination and imaging filter.

FIG. 7 illustrates travel of light from a light source assembly to an imaging sensor.

FIG. 8A illustrates retinal illumination beams from a pair of opposing outward facing light sources.

FIG. 8B illustrates light launch from a light source assembly pre-combined through optical fibers, split and redistributed.

FIG. 8C illustrates light launch from a light source assembly through optical fiber and individually transmitted.

FIG. 8D illustrates light launch from a light source assembly through individual fibers utilising the entire available launch positions.

FIG. 9 illustrates light beam paths through an annular filter assembly.

FIG. 10A illustrates an annular cross polarizer filter assembly.

FIG. 10B is an exploded view of an example filter assembly for use in an apparatus for multi-spectral retinal imaging.

FIG. 11 illustrates exchange of filter-pair assemblies in a multi-spectral imaging apparatus.

FIG. 12A illustrates an embodiment of an annular filter cassette comprising multiple filter assemblies.

FIG. 12B illustrates an embodiment of a linear filter cassette comprising multiple filter assemblies.

FIG. 13 illustrates a method of obtaining wide field of view retinal imaging including fundus autofluorescence.

FIG. 14 illustrates a retinal image with wide field of view based on combined retinal images.

FIG. 15 illustrates the casing of an apparatus for multi-spectral retinal imaging with wide field of view including multi-spectral fundus auto fluorescence.

FIG. 16 is a comparison of long multi-spectral imaging with autofluorescence.

FIG. 17 provides example retinal images for a variety of retinal diseases using multi-spectral imaging.

FIG. 18 illustrates a multimodal image case comparing fundus imaging with MSI images and MSI-FAF at 660 nm.

FIG. 19 shows multi-spectral retinal imaging with autofluorescence.

FIG. 20 shows a comparison between a color fundus image compared to multi-spectral imaging with fundus autofluorescence at 660 nm.

FIG. 21 provides examples of fundus autofluorescence compared to multi-spectral imaging retinal images.

FIG. 22 provides more examples of fundus autofluorescence compared to multi-spectral imaging retinal images.

FIG. 23 provides a series of multi-spectral images taken at different wavelengths.

### DETAILED DESCRIPTION OF THE INVENTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprise” and any of its derivatives (e.g. comprises, comprising) as used in this specification is to be taken to be inclusive of features to which it refers, and is not meant to exclude the presence of any additional features unless otherwise stated or implied. The term “comprising” as used herein will also be understood to mean that the list following is non-exhaustive and may or may not include any other additional suitable items, for example one or more further feature(s), component(s) and/or element(s) as appropriate.

As used herein, the terms “comprising”, “having”, “including”, and “containing,” and grammatical variations thereof, are inclusive or open-ended and do not exclude additional, unrecited elements and/or method steps. A composition, device, article, system, use, or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to. The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment”, and “for example” is intended to illustrate or denote aspects, embodiments, variations, elements or features relating to the invention and not intended to limit the scope of the invention.

As used herein, the terms “connect” and “connected” refer to any direct or indirect physical association between elements or features of the present disclosure. Accordingly, these terms may be understood to denote elements or features that are partly or completely contained within one another, attached, coupled, disposed on, joined together, in communication with, operatively associated with, etc., even if there are other elements or features intervening between the elements or features described as being connected.

Herein is described an apparatus and method for obtaining multi-spectral retinal imaging with wide field of view including multi-spectral fundus auto fluorescence. The present apparatus has a light source assembly for retinal illumination for wide field of view retinal imaging. Wide field of view images can be obtained at multiple wavelengths, also referred to as multi-spectral images, including autofluorescence images, to provide effective, efficient, and extended retinal disease identification, monitoring and diagnostics. The presently described apparatus assembly follows general principles of optical imaging but has a unique design implementation to enable a broad range of wavelengths, including fluorescence wavelengths, to be accurately imaged, effectively at the same time. The present apparatus provides a compact, simplified design, with high performance to provide quality wide field retinal images.

A multi-spectral retinal ophthalmoscope as presently described can provide illumination to the retina for imaging of multiple individual wavelengths across a wide wavelength range from visible to near infra-red (NIR). The presently described multi-spectral retinal imaging (MSI) technique and apparatus has proven to be an effective tool for enhanced visual identification of many of the above-mentioned biomarkers compared with conventional color fundus imaging. Various clinical investigations have demonstrated the effectiveness of MSI for early detection and diagnosis of a variety of eye conditions and diseases. A method of using a dedicated multispectral fundus autofluorescence (FAF) camera using excitation light wavelength to stimulate the retina to obtain fundus auto fluorescence images on one or multiple spectra is also described. The present apparatus comprising multispectral light sources, light launching optics, groups of lenses to deliver excitation light to retina of human eyes and collect fluorescence emissions from the retina, optical filters selectable to match the excitation wavelength, an image sensor, and control electronics can be used for the purpose of obtaining multispectral fundus and auto-fluorescence retinal images.

The retina of an average adult human has a concave spherical shape with a circumference arch span of approximately 20 mm from the center of the eye and spherical diameter of 22 mm. Traditional fundas camera has 40 to 45 degrees field of view (FOV) of the retina due to several limitations. Many retinal diseases including glaucoma, retinal tears, Stargardts disease, melanoma to name few, may appear and affect peripheral areas of the retina, driving the need for an ability to easily image and observe a wide field of view of the retina. There are several challenges involved in broadening the FOV beyond 45 degrees. Extending the FOV to 120 degrees in the presently described apparatus for multi-spectral retinal imaging is achieved through optics design as described herein. However, the wider field of view comes with a set of illumination and imaging challenges such as light distribution, sensing coverage, and Purkinje reflections. This disclosure provides several unique methods as follows to overcome the discrepancies associated with the wide field of view and extend it to 120 degrees as described below.

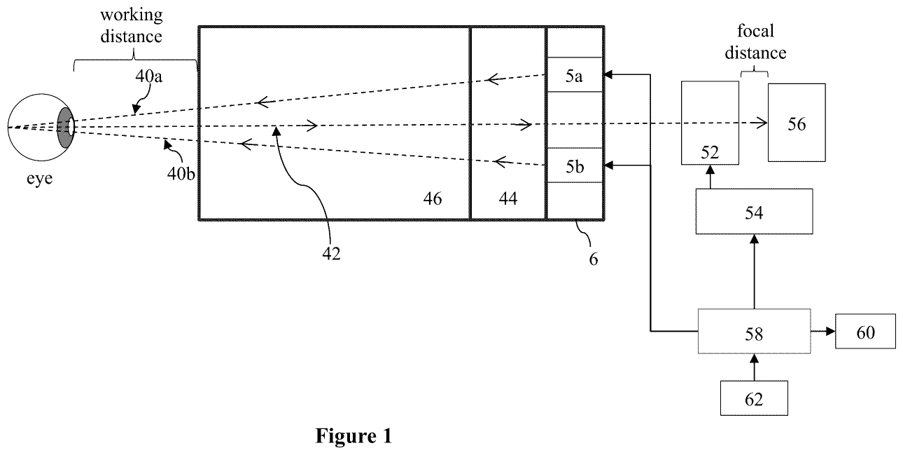

FIG. 1 is a perspective view of an apparatus for multi-spectral retinal imaging with a wide field of view. Multi-spectral fundus autofluorescence (MS-FAF) capability may be integrated with a multispectral fundus camera, resulting in a multifunctional multispectral ophthalmoscope. The presently described multi-spectral fundus autofluorescence (MS-FAF) imaging system provides a light imaging path realized by three sections of well aligned optics assemblies: the front optics assembly 46 to transform light beams from small numerical aperture to larger aperture and vice versa in the opposite direction; the beam management assembly 44 to allow proper light beam forming and bidirectional light isolation; and the rear optical assembly or imaging optics assembly 52 that compensates for diffractions and other errors as well as launches the imaging light beam correctly to a given imaging sensor 56. Actuator 54 is capable of moving an element inside imaging optics assembly 52 to achieve focus adjustment so that the imaging sensor 56 is at the optimal focal distance for a given accommodation of the eye under test.

The sets of light illumination assemblies, 5a, 5b, comprise a plurality of light sources distributed around a set of lenses in a light source assembly 6 that forms a beam management optics assembly, which is part of the entire imaging path and originator of the illumination light in the presently described apparatus for multi-spectral retinal imaging. Each light source in illumination assemblies, 5a, 5b can comprise one or more of a light emitting diode (LED), array of LEDs, fiber-optic light source, laser, hyper spectral laser, wide-spectral tunable laser, or other suitable light source capable of sufficient illumination to illuminate the retina for imaging. The light source can also be of several discrete light emitting diodes (LEDs) or lasers with the same or different wavelengths or can comprise multiple optical fibers that bring light from a single or a group of such LEDs or lasers simultaneously. Furthermore, the individual light sources may be combined through wave division multiplexing or high-pass or low-pass combiners instead of mirrors. In one example, LEDs can be used as a source of illumination through shaping optics. In addition, multiple LEDs or an LED array such as a quad LED array can be used in the same location as a single LED is as the light source. In another example, a broadband laser, hyper-spectral laser, or a broadband tunable laser may also be used as light source. Light can also be pre-combined and launch into an optical fiber or a bundle of fibers and be brought to the same said location or a multiple of said locations to function as the light source.

Emitted light from the light sources in each illumination assembly, 5a, 5b is launched into the beam management assembly 44, which is the middle section of the optical assembly with the emission orientation towards the eye and distributed around within a pre-defined ring. Positioning of a plurality of illumination assemblies around a ring in light source assembly 6 allows light from a plurality of locations on the same plane and with the same radius to the detection axis to be launched, providing a wide and adequate illumination of the retina. Illumination light beams 40a, 40b illustrate the angle of the launched illumination light relative to the detection axis for the imaging light beam 42. The received imaging light beam from the opposite direction originating from the eye is confined into a disc shape within the ring of the light source assembly 6. This allows at this proximate location a clear separation between the illumination light beams and the collected imaging light beam, hence permits the manipulation or filtering of the illumination light beam and imaging light beam separately and respectively. For example, the spectral of the illumination light may be re-shaped through a bandpass, long-pass, or short pass filter; or the illumination light is passing through a wideband polarizer, and the imaging light is passing through another polarizer with orthogonal orientation to the illumination polarizer to suppress retroreflections. Another example is the illumination light is re-shaped through a spectral filter for fluorescent excitation and the imaging light beam is filtered through a long pass filter to pick up the corresponding auto fluorescent responses.

An imaging sensor 56 with adequate resolution and with high sensitivity and low noise is utilized to detect reflected imaging light from the eye's retina. Commercial products such as SONY IMX542 or IMX387 CMOS sensors will work well in such applications. Once received by the imaging sensor 56, the collected retinal images are moved by control module 58 for processing, storing, viewing by display 60. Input device 62 connected to control module 58 for apparatus operating, tuning and control. Focus adjustment is realized by control module 58, which provides electronic control and coordination among the different modules including illumination assemblies 5a, 5b, imaging sensor 56, and other actuators in the apparatus used to control distances between optical components or move optical filter assemblies. Control module 58 with embedded programming can be operated by a user through an interface at input device 62, which optionally has a keyboard, mouse, or other input device. The control module 58 comprises one or more connected computers or computing systems, wired or wirelessly connected.

In an embodiment, control module 58 is a small single-board computer, an embedded computing system, or a computing platform running a set of specific application control software algorithm. A control signal can be sent from control module 58 through dedicated interface ports, with the instructions and synchronization timing signals to each of the other modules in the system. For example, image capture requires timely coordinated actions commanded by the control module. For each image to capture a specific illumination or fluorescent wavelength, the control module 58 needs to prepare the corresponding light source with the identified wavelength, place the appropriate imaging light filter and fluorescence filter in the light path, and set the image sensor ready. Once the apparatus is in ready mode, the control module 58 will send the command to start light source flashing and a trigger signal to the image sensor will be sent and image sensor will capture the image as the light source is flashing. This trigger synchronized image capture allows the apparatus to maximize the efficiency of the imaging and reduce excess light exposure to the eye. The control module 58 is also responsible for collecting the captured images, storing and uploading them to a designated and secured data management site. Control module 58 is also used to provide electronic control and coordination among the different modules including illumination assemblies 5a, 5b, imaging sensors 56, and other actuators.

Conventional fundus camera uses a white light source and collect an all-spectrum combined color image. In contrast, a multi-spectral retinal ophthalmoscope uses light source with discrete wavelength spectra across a wavelength range of about 525 nm (green) to 850 nm (NIR) to progressively examine the different layers of the retina and choroid, with the longer wavelengths penetrating deeper into the structures of the eye. Each monochromatic spectral slice represents successive images of the fundus as targeted and deliberately selected different wavelength differentially reflect, scatter, and absorb deeper into the posterior pole, enhancing visibility of disease biomarkers in the retinal and choroidal layer. Multi-spectral imaging optionally together with autofluorescence imaging enhances the visualization of the entire posterior pole of the eye, covering from the internal limiting membrane (ILM) through to the choroid, highlighting the retinal pigment epithelium (RPE).

The present multi-spectral retinal imaging apparatus can be used by optometrists and ophthalmologists to obtain a series of wide field retinal images of a patient eye under test. The apparatus can also be referred to as a multiwavelength ophthalmoscope or multi-spectral fundas camera. Each of the images are collected at a specific center wavelength at at least two inclination angle s to the detection axis to achieve a wide field of view retinal image. This is achieved by illuminating the retina with a specific wavelength and then capturing the image. Two or multiple illumination light sources at different light source position of the same wavelength can be controlled to flash at the same time to provide stronger illumination. Each illumination light source can be, for example, individual discrete light emitting diodes (LEDs), an array of LEDs, a hyper spectral laser, wide spectral tunable laser, or light transmitted from one of these sources through a fiber optic cable to light source assembly 6. Images are captured through a set of lenses including objective lens 38 and lenses in imaging optics assembly 52, forming a quality imaging system and then by using a monochromatic digital image sensor. The image capturing by the imaging sensor 56 is synchronized with light sources in the light source assembly 6 by a trigger from control module 58. This allows the apparatus for multi-spectral retinal imaging to maximize the efficiency of the imaging and reduce extra light exposure.

Since the light direction of illumination to the retina and collected reflective images of the retina are in opposite directions, there are several methods that can be used to separate illumination light from the reflected imaging light as much as possible to avoid direct reflection of illumination light back into the image sensor from multiple lens surfaces and front of the eye. In one control method, the imaging sensor is set in a trigger mode, the light source flash pulses are sent to the image sensor, and each light flash pulse will trigger the sensor to capture an image for the duration of the flash. In another control method the light source control is set in a trigger mode, and each time the image sensor is ready to capture an image the control system will send a trigger pulse to the light source control electronics, which in turn provides a control signal to flash the light source, one or multiple at a time, for a preset duration in concert with each image sensor capture. The acquisition time for multi-spectral imaging is typically less than 40 milliseconds, and for FAF the acquisition time is typically less than 250 milliseconds. Preferably, the light illumination time for each image acquisition is between about 10 and 250 milliseconds. Short image acquisition times substantially minimizes any involuntary micro saccadic movements of the eye from blurring the image. The light source flash duration time is set by the control module 58 and is set for each light source individually and for each flash. The control module can also operate in auto exposure mode. During focusing retinal image data is collected which allows calculation of the amount of energy required for imaging a specific retina pigmentary density. The darker the retina, the longer exposure is required, and this will allow the control module to proportionally adjust exposure for all wavelengths from short to near infrared (NIR).

The presently described multi-spectral retinal ophthalmoscope, or multi-spectral FAF retinal imaging apparatus, enables wavelength selection and spectral filtering to produce specific excitation or illumination light to illuminate the retina and to collect a corresponding retina image including within the auto fluorescence spectral range. For fluorescence imaging in particular, multi-spectral fundus autofluorescence (FAF) imaging can be achieved through the selection and paring of the excitation light source wavelength and the spectral filtering of a FAF filter. It covers excitation blue (480) FAF, green (550) FAF, amber (600 nm) FAF, deep Red (660) FAF, NIR1 (780) FAF, and NIR2 (810) FAF, etc. and serves as an example of multi spectral Auto Fluorescence selection. A typical FAF is achieved by illuminating the retina with a specific wavelength and then capturing an image from the retinal with the correspondence fluorescence emission. The illumination or excitation light source for each specific wavelength FAF can be a series of individual discrete light sources such as light emitting diodes (LEDs) of the same spectral, individual lasers, a single hyper spectral laser, or a wide spectral tunable laser. Fluorescence images can be captured through the same set of lenses including objective lens 38 and the lenses and optical components in imaging optics assembly 52, forming a quality imaging system with an optical filter to screen out non-fluorescence lights such as the excitation light or stray light. In one preferred embodiment, the combination of spectral filtering of illumination light and imaging light together with different spectral filters for illumination light and imaging light respectively in the similar arrangement as assembly 22 with the same monochromatic digital imaging sensor 56 providing high quality FAF images.

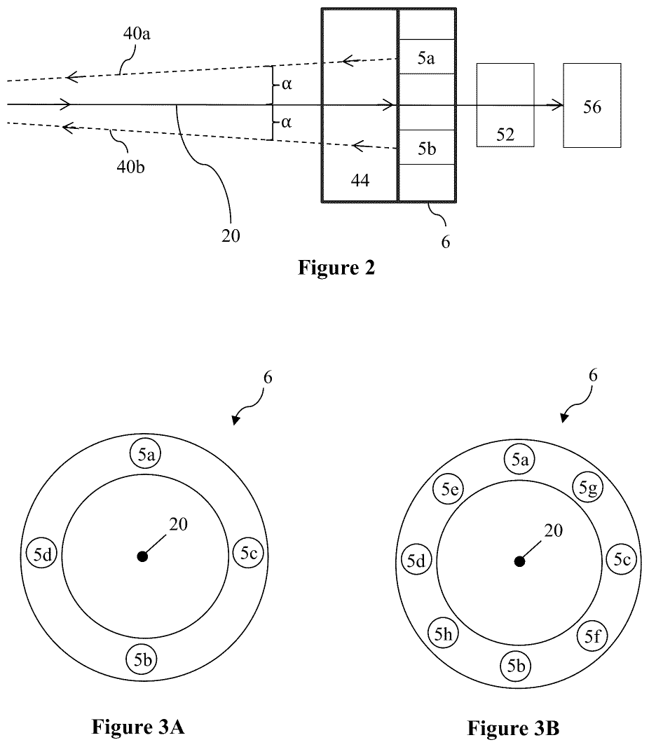

FIG. 2 is a cross-sectional schematic of a multi-spectral retinal imaging apparatus with a wide field of view. Illumination assemblies 5a, 5b in light source assembly 6 can launch light from different locations relative to the detection axis 20. Each illumination assembly comprises one or more light sources together with light guiding devices such as one or more lenses, mirrors, and dichroic mirrors. Illumination light beams 40a, 40b originating from illumination assemblies 5a, 5b, respectively, can be redirected towards the desired launching direction at an inclination angle α relative to the detection axis. Imaging light then travels back from the eye along detection axis 20 as an imaging light beam to imaging optics assembly 52 and imaging sensor 56.

FIGS. 3A and 3B are cross-sectional views of a light source assembly 6 for a multi-spectral retinal imaging apparatus with a wide field of view, where FIG. 3A illustrates a light source assembly with four illumination assemblies 5a, 5b, 5c, 5d, and FIG. 3B illustrates a light source assembly with eight illumination assemblies 5a, 5b, 5c, 5d, 5e, 5f, 5g, 5h. In a preferably embodiment, the illumination assemblies are positioned in a ring in light source assembly 6, with an aperture in the ring open so as not to interfere with the imaging light beam traveling along detection axis 20. Pre-forming the illumination light beam in a ring shape allows the physical separation between the illumination light path and the incoming imaging light path, therefore minimizes the overlapping and reduces the amount of direct and indirect reflections.

Different light source arrangements can be done with respect to the main optical assembly body of light source assembly 6. Illumination light beams can be launched from any of the illumination assemblies directly towards the pre-determined direction into the beam management system.

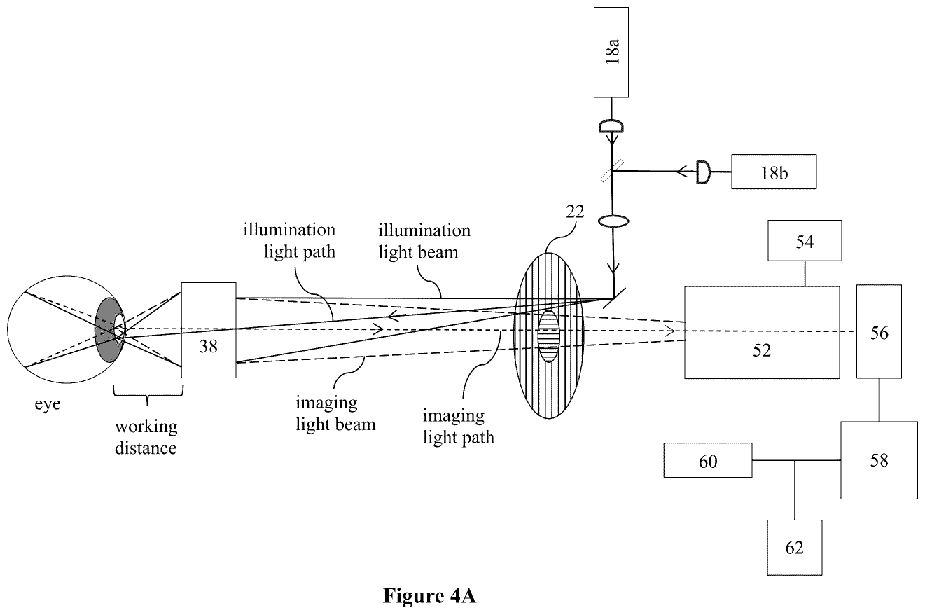

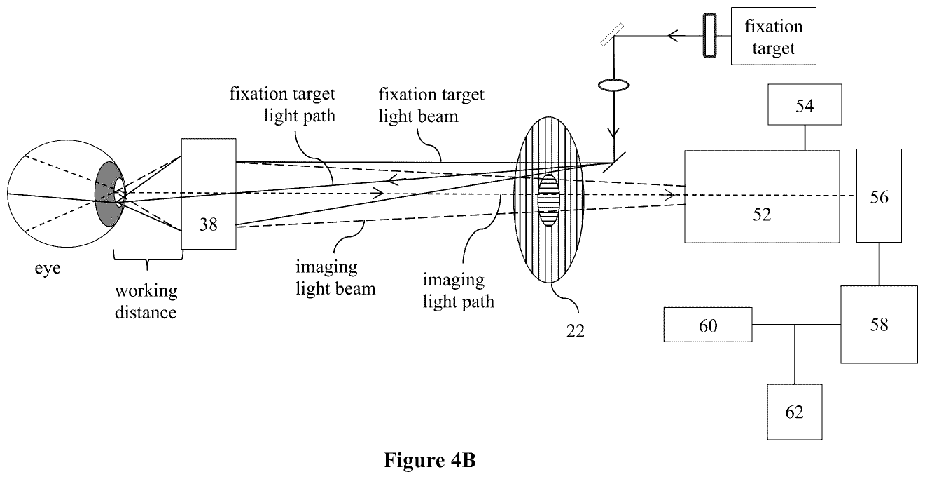

FIG. 4A is a cross-sectional schematic of a beam path from an illuminated light source in a light source assembly and light shaping objective lens. Light sources 18a, 18b provide illumination light, which is directed to filter assembly 22 via suitable light shaping and light directing optical components to provide illumination light beams in an illumination light path. Light sources 18a, 18b may light the retina with continuous illumination or with pulsed illumination. Filter assembly 22 shown comprises an annular illumination filter which filters illumination light. Objective lens 38 receives the illumination light beams and directs the light onto the eye, forming the light beam waist on the cornea. Through the eye lens, the light beam expands to illuminates the retina with 120-degree field. This illuminated 120-degree filed of the retina forms a retinal image whose imaging light beams transmit through the eye pupil and then the objective lens 38 aligned in an imaging light path of the instrument. They are further directed through an imaging filter in filter assembly 22 to imaging optics assembly 52 and imaging sensor 56. Actuator 54 provides light focus adjustment to one or more optical components in imaging optics assembly 52. Also shown are control module 58, display 60, and input device 62. The illumination to realize a wide angle and even degree of lighting on the retina is realized with placement of light source and special optical lens system design.

Image capture is based on an imaging sensor 56 with resolution, performance, and capture rate that match the requirement and quality of the imaging needs. For pulsed illumination, the image sensor is synchronized in capture with the illumination pulsing. Images captured are subsequently stored electronically on a non-volatile storage medium and may be processed to compensate for various imperfections in the optical system, and to spatially align them. Additionally, common defects can be corrected in the collected images, including but not limited to unwanted reflections, unwanted scatterings, and uneven illuminations. The images can then be analyzed in different ways or using different methods to optimize the ease of interpretation by the ophthalmologist. Furthermore, the imagine optics can be adjusted so that the projected retinal image is in focus on the image sensor, in other words, to cause the image sensor on the focal plane to accommodate the fact that each eye from each person requires different accommodation.

There are multiple ways to implement focus adjustment through optical design. One is to have a set of optical lenses with at least one of which movable along the optical axis, so that the imaging sensor 56 can remain in a fixed position with respect to the entire optics. Another way is to have a fixed optics but moving the imaging sensor 56 towards or away from the optics so that it can be on the focal plane of the retinal image.

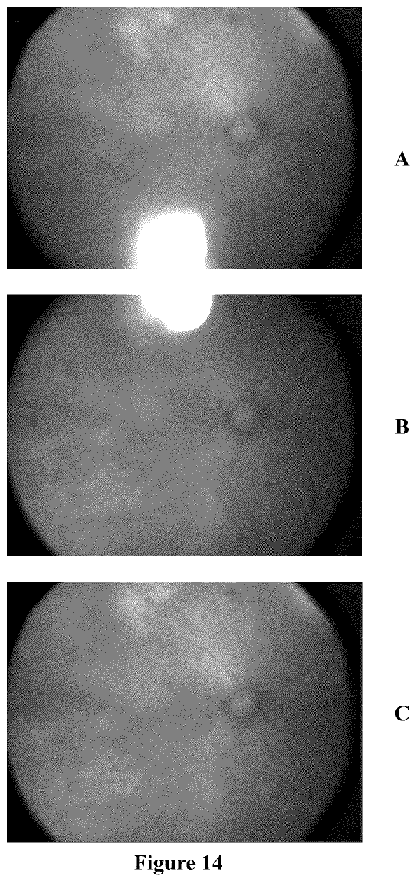

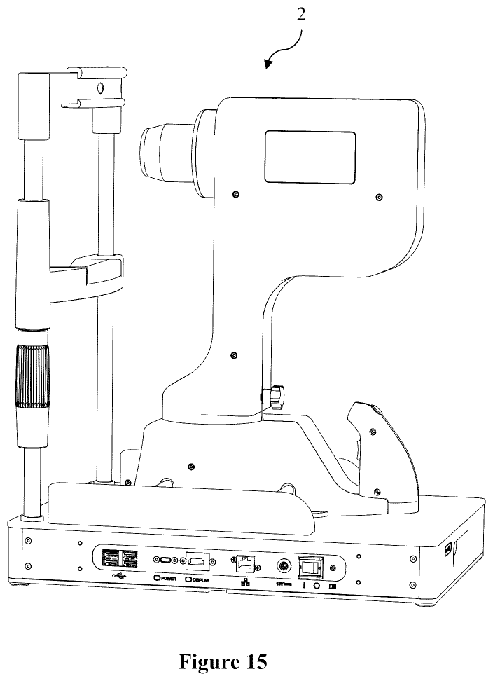

One significant challenge with imaging the retina with a wide field of view beyond 45-degree is the presence of Purkinje reflections. These reflections are from the front and back of the cornea and lens surfaces. To suppress surface retro-reflections a cross-polarizer filter assembly 22 can be used which helps to reduce the reflections. Another method to remove Purkinje reflections, as presently described, can be done by using two light sources of the same wavelength and physically placed opposite to each other in a light source ring, as shown in FIGS. 3A and 3B. The Purkinje reflections from each such paired light sources will appear on the opposite part of the retinal image taken with each light source independently. Taking two separate retinal images with one of the paired light sources illuminating results in one strong Purkinje reflection in each image and in different parts of each two images. This is shown in FIG. 14. Each pair of images can then be processed to remove the strong Purkinje reflections and stitched or montaged together to form a full wide field retina image. This can be done using any commercial or proprietary panoramic image processing software and known techniques.

FIG. 4B is a cross-sectional schematic of a beam path from an illuminated light source with a fixation target 78. During a retinal imaging session patients need to maintain a stable gaze to acquire good quality images. The object that the patient gazes at is called a fixation target. The fixation target provides an image for a patient to focus on during image acquisition. In general, a fixation target can be, an external object or specific point of an object. The fixation target can also be a light spot or pattern inside the ophthalmoscope as in FIG. 4B that the patient can see when their eye is in position to be imaged and as this eye is looking into the imaging apparatus. The fixation target provides a back-lit pattern, whose light is directed to filter assembly 22 via suitable light shaping and light directing optical components to provide a fixation target light beam in a fixation target light path. The light source providing the fixation target may light the retina with continuous illumination or with pulsed illumination. Filter assembly 22 shown comprises an annular illumination filter which filters fixation target light. Objective lens 38 receives the fixation target light beams and directs the light onto the eye and forming an image of the fixation target on the retina, therefore being seen as an object by the eye. Also shown are control module 58, display 60, and input device 62. In an embodiment, the plano-convex lens in front of one of the illumination light sources in the illumination assembly can be replaced with a different lens or another optical component so that the fixation target can be imaged on the retina such that it can be seen clearly by the patient. In an embodiment, the fixation target 78 is a light spot constructed such that it replaces the illumination light source such as 18a, or 18b, etc. and placed in the position where an illumination light source is.

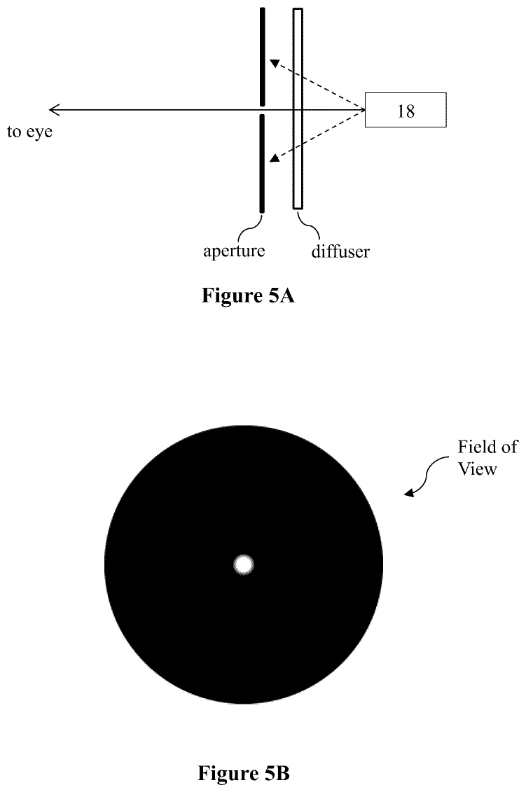

FIG. 5A is a schematic of a fixation target assembly indicating the light paths to the eye. It consists of a light source 18 of a certain wavelength to provide back lighting. In front of the light source 18 is a diffuser to make the back lighting an even, smooth and non-directional light. This light can then provide back lighting for the object panel, or in the example shown in FIG. 5A, a small dot created by a dark panel with a pinhole aperture back lit by a smooth and deem light. FIG. 5B is an illustration of fixation target as perceived by a patient. In order for a fixation target to be projected onto the patient's retina via an illumination channel, it is necessary that the image be in reasonable focus. Small target sizes can be used maintain relatively small foot-prints observed by the patient and the location of the projected target image on the retina can be adjusted simply by off-setting the target.

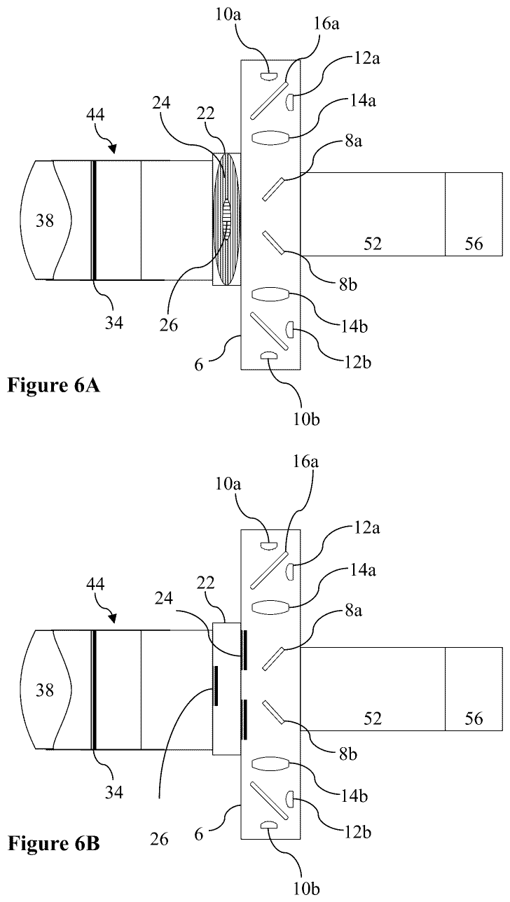

FIG. 6A illustrates an annular polarizer with inner polarization disc and outer polarization ring with a light source assembly for multi-spectral imaging. The polarizer assembly 22 shown has a linear outer polarizer 24 to receive illumination light beams directed to the eye, and a linear inner polarizer 26 for receiving imaging light beams from the eye. The ring shape allows a separation between the illumination light beam and the incoming imaging light beam, therefore minimizes the overlapping and reduces the amount of direct and indirect reflections. The inner polarizer 26 is at a different polarization from the outer polarizer 24 and with polarization direction oriented orthogonal to the outer polarizer 24, hence forming a cross-polarization condition to limit retro reflections and specular reflections of illumination light beams back into the imaging sensor 56. In one embodiment of the polarizer assembly, the outer polarizer which is illumination light filter 24 can be a polarization ring and the which is imaging light filter 26 can be a polarization disc which is coplanar with the polarization ring. The outer polarizer which is illumination light filter 24 and inner polarizer which is imaging light filter 26 may be, for example, a thin layer of aluminum MicroWires attached to a glass substrate or layered between two Fused Silica windows. They can also be lithographically deposited on glass substrates. These metal wires are typically 100 nm to several microns apart.

Light source assembly 6 comprises a light source assembly with a plurality of illumination assemblies. A pair of light source assemblies is shown. Each illumination assembly has a downward facing light source at shorter wavelengths 10a, 10b, an outward facing light source 12a, 12b, at longer wavelengths and a short-pass dichroic mirror 16a, 16b to combine the light paths of two light sources of different wavelengths by letting the shorter wavelength light transmit though directly and by reflecting the longer wavelength light by 90 degrees, hence bringing the light paths from 10a and 12a to superimpose and towards the same propagation direction.

A dichromatic mirror, also known as a dual-band or dual-wavelength mirror, is a mirror that reflects or transmits light differently at two different wavelengths. Dichroic mirrors are often made of multilayer dielectric thin films on a transparent or glass substrate. The two most common types of dichroic mirrors are short-pass and long-pass Mirrors. A short-pass mirror is one which has a high transmittance at short wavelengths and high reflectance at longer wavelengths. There is a transition wavelength, such that any light with shorter wavelength will transmit through and light with longer wavelength than the transition wavelength will not be transmitted, rather they will be reflected. The transition wavelength is also referred to as the cut-off wavelength of the dichroic mirror. In an example, a short pass dichroic mirror with a cutoff wavelength at 660 nm may have a transmission band from 450 nm to 655 nm and reflection band from 665 nm to 900 nm. A long pass dichroic mirror works in exactly the same way except the longer wavelengths of light will transmit straight through and shorter wavelengths of light will be reflected. A wide selection of short pass and long pass dichroic mirrors at different cutoff wavelengths are available. Other features of dichroic mirrors can include custom parameters such a specific cutoff wavelength, transmission or reflection spectral ranges, transmission or reflection losses etc. Typically, the cutoff wavelength of a dichroic mirror is selected to correspond to the peak wavelengths and spectral widths of the light sources, i.e. the emitted spectra of the downward facing light sources and outward facing light sources, to optimize the throughput of both reflection and transmission of the emitted light. Further, the orientation and position of the dichroic optical mirror, and light sources, are configured together so that the light can be optically combined in the illumination assembly. The cutoff wavelength may also be shifted dynamically by using a tunable dichroic mirror or filter. The shifting of the cutoff wavelength can be achieved, for example, mechanically adjusting the alignment angle of the mirror, i.e. changing the incidence angle of the light.

Each light source in light source assembly 6 can be illuminated in sequence to obtain a plurality of retinal images at different wavelengths to provide a plurality of wide field of view retinal images. Light shaping lens 14a, 14b modifies the illumination beam divergence and focal length. The light shaping lens can be, for example, an equi-convex or bi-convex lens. Directional optical components 8a, 8b direct the illumination light beam towards objective lens 38 at an inclination angle relative to the detection axis through illumination light filter 24 in the filter assembly 22 which also has an imaging light filter 26. Beam management assembly 44 has an optional photosensor 34 to measure light travelling from the light source assembly 6 to the retina and is connected to a control circuit to ensure that the light intensity reaching the retina is within safe limits. Objective lens 38 provides beam direction to and from the retina. Light returning from the retina follows the imaging light path on the detection axis through imaging optics assembly 52 to imaging sensor 56.

FIG. 6B is a cross-sectional view of a polarizer assembly with offset illumination and imaging filter. Light source assembly 6 comprises a light source assembly with a plurality of illumination assemblies. Each illumination assembly has a downward facing light source 10a, 10b, an outward facing light source 12a, 12b, and a dichroic mirror 16a, 16b to direct light from the illuminated light source. Each light source in light source assembly 6 can be illuminated in sequence to obtain a plurality of retinal images at different wavelengths to provide a plurality of wide field of view retinal images. Light shaping lens 14a, 14b modifies the illumination beam and shapes the illumination light from downward facing light sources 10a, 10b and outward facing light sources 12a, 12b and can be, for example, an equi-convex or bi-convex lens. Directional optical components 8a, 8b direct the illumination light beam towards objective lens 38 and at an inclination angle relative to the detection axis through illumination light filter 24 in the filter assembly 22 which also has an imaging light filter 26. Filter assembly 22 is shown with an illumination light filter 24 and imaging light 26 that are offset from one another along the detection axis. Beam management assembly 44 has an optional photosensor 34 to measure light travelling from the light source assembly 6 to the retina and is connected to a control circuit to ensure that the light intensity reaching the retina is within safe limits. Objective lens 38 provides beam direction to and from the retina. Light returning from the retina follows the imaging light path on the detection axis through imaging optics assembly 52 to imaging sensor 56.

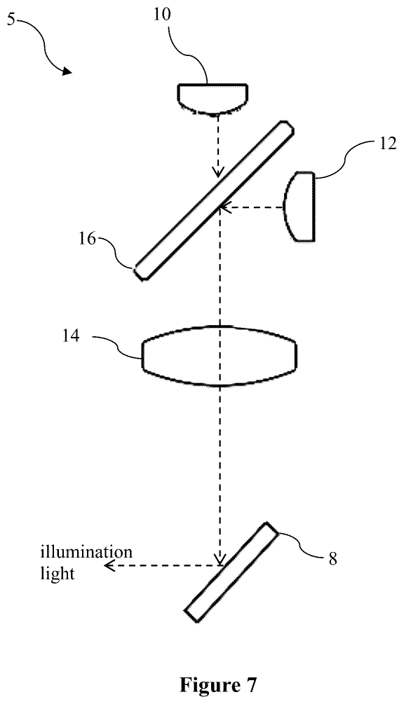

FIG. 7 illustrates travel of light from a light source assembly 5 towards an objective lens. Light can be directed from downward facing light source 10 or outward facing light source 12 toward a dichroic mirror 16. The dichroic mirror 16 can be a dichroic optical filter serving as mirror for the outward facing light source 12 or be transparent to downward facing light source 10 to direct light toward light shaping lens 14. In an embodiment, a short-pass filter can be used to serve as a combiner to bring light paths of different wavelengths together. In which case the light source 10 is a shorter wavelength light source, for example shorter than 660 nm and light source 12 is a longer wavelength light source, for example longer than 660 nm. Alternatively, the position of the light source with short wavelength and longer wavelength may be interchanged, such that the downward facing light source has a peak wavelength longer than 660 nm and outward facing light source has a peak wavelength shorter than 660 nm. In this case, a long-pass dichroic mirror is used to allow more efficient combination of transmitting and reflecting light. Directional optical component 8 receives light from light shaping lens 14, which can be, for example, an equi-convex or bi-convex lens, and direct the illumination light beam on to a beam management assembly.

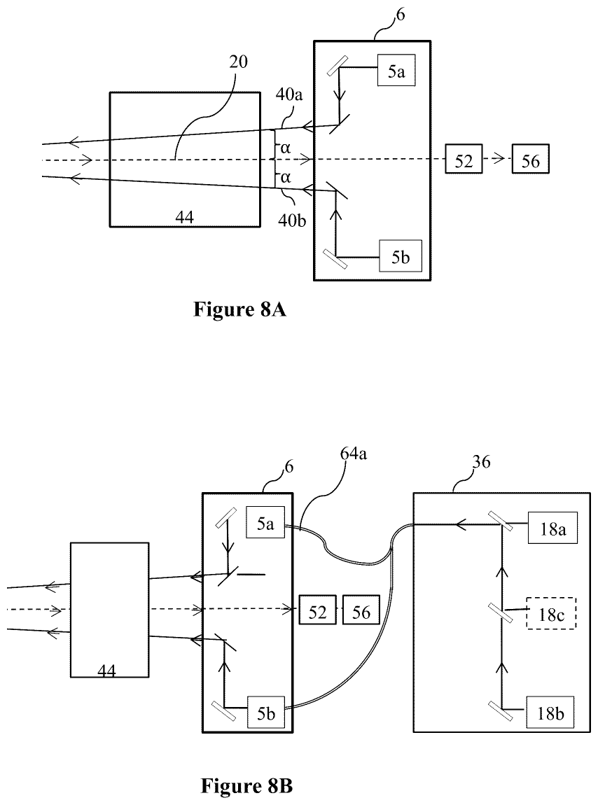

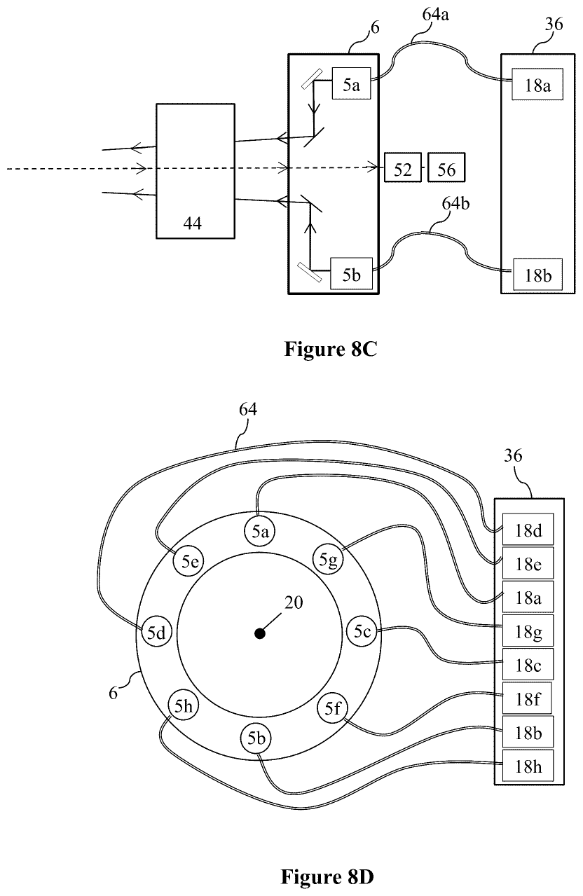

FIG. 8A illustrates retinal illumination beams from a pair of opposing outwards facing light sources. For a pair of opposed illumination assemblies 5a, 5b in light source assembly 6, a single image can be taken from an illumination beam from each of the two opposing illumination assemblies 5a, 5b. After imaging light has been received by imaging optics assembly 52 and imaging sensor 56, the two obtained images can be registered (aligned), processed, and montaged to form a composite image removing Purkinje reflections altogether. In particular, a first image taken from first illumination assembly 5a with illumination light beam 40a provides a first retinal illumination beam at a first inclination angle α relative to the detection axis, and a second image taken from a second illumination assembly 5b with illumination light beam 40b, which is on the opposite side of illumination assembly 6 from first illumination assembly 5a, provides a second retinal illumination beam at a second angle −α relative to the detection axis, which is shown overlapping with orthogonal light beams from illumination assemblies 5c, 5d. It is noted that the first illumination beam 40a and second illumination beam 40b are coplanar as they are on opposite sides of the illumination assembly, as well as coplanar to the detection axis 20. A similar set of images can be taken from illumination assemblies 5c, 5d, which are orthogonal to illumination assemblies 5a, 5b, shown in FIG. 3A. Beam management assembly 44 provides beam shaping and beam direction to illumination light beams 40a, 40b and to the imaging light from the retina to the imaging optics assembly and imaging sensor. Beam management assembly 44 can comprise an objective lens such as an equi-convex lens and/or bi-convex lens, as well as other lenses and light shaping devices. In one embodiment, light can be launched into the illumination light path through a combination of a plano-convex and a bi-convex lens sets. Other lenses such as Fresnel lenses can also be used for LED light shaping. Other optical elements can be used to further improve light quality such as, for example, microlens arrays and/or diffusors to improve light uniformity, and band-pass filters to shape light beam spectral. In other embodiments, gradient index micro (GRIN) lenses or ball lens may be used to couple light into optical fibers.

FIG. 8B illustrates light launch from a light source assembly through optical fibers. In this embodiment illumination light from any one of illumination assemblies 5a, 5b can be launched directly towards the pre-determined direction from light source assembly 6. Light supplied to originates from a remote light generation assembly 36 having one or more light sources 18a, 18b, 18c, etc. Lights from light sources of different wavelengths can be combined using wavelength multiplexer and coupled into a single strand of optical fiber or a bundle of optical fibers. The single strand of fiber or fiber bundles can then be split to two or more fibers or fiber bundles, each feed into a designated illumination location such as 5a, 5b, etc. in illumination assembly 6 as seen in FIG. 8D. The single strand of fiber is typically a large area multi-mode fiber with core diameter 1 mm or larger and with a numerical aperture (NA) of 0.25 or higher. Light returning from the eye is received at imaging optics assembly 52 and imaging sensor 56.

FIG. 8C illustrates an alternative fiber illumination scheme with light launched from a light source assembly through optical fiber and individually transmitted. Light generation assembly 36 can comprise one or more wide spectral tunable laser light source, connected to each of illumination assemblies 5a, 5b through one or more optical fiber. In the embodiment shown, optical fibers 64a, 64b transmit light from light sources 18a, 18b in light generation assembly 36 to illumination assemblies 5a, 5b respectively. Each optical fiber can feed into a designated or corresponding illumination location for any or all locations in illumination assembly 6 as seen in FIG. 8D. The transmitted light is directed toward the eye through beam management assembly 44 and light returning from the eye is received at imaging optics assembly 52 and imaging sensor 56.

FIG. 8D illustrates light launch from a light source assembly through individual fibers utilising the entire available launch positions. Light generation assembly 36, which can be remote from light source assembly 6, can connect light sources 18a-18h to illumination assemblies 5a-5h through a plurality of optical fibers 64. Light can thereby be generated remotely but may be launched from different orientations or locations on light source assembly 6. For example, radial orientation and with the help of a set of mirrors and/or other optical components, the illumination light beams can be redirected towards the desired launching direction. Pre-forming the illumination light beams in a ring shape allows the physical separation between the illumination light path and the incoming imaging light path, therefore minimizes the overlapping and reduces the amount of direct and indirect reflections. A ring shape of the light source assembly 6 also allows a separation between the illumination light beam and the incoming imaging light beam, therefore minimizes the overlapping and reduces the amount of direct and indirect reflections. The light sources 18a-18h can be of several discrete light emitting diodes (LEDs) or lasers with the same or different wavelengths. Light can also be transmitted by one or multiple of optical fibers to bring lights from a single or a group of such LEDs or lasers simultaneously. Individual lights may also be combined through wave division multiplexing or high-pass or low-pass combiners instead of mirrors. Light sources may also light the retina with continuous illumination or with pulsed illumination.

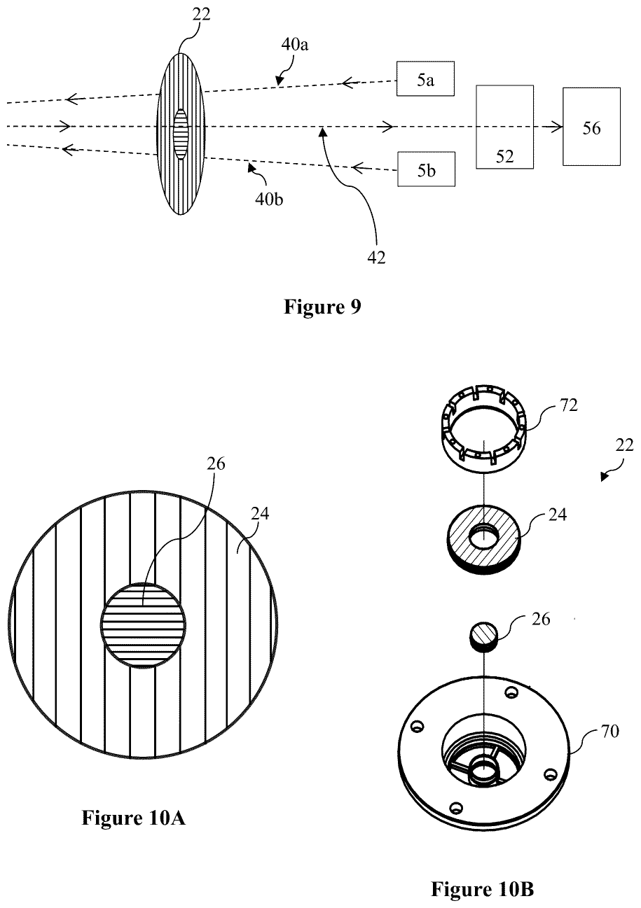

FIG. 9 illustrates light beam paths through an annular filter assembly. Illumination assemblies 5a, 5b are illuminated sequentially to provide two retinal images at the same illumination and imaging wavelength which can be stitched together to provide a single wide field of view retinal image. The time delay between illuminations of orthogonal illumination assemblies 5a, 5b can be, for example, 10-50 milliseconds. In an illumination of illumination assembly 5a, light beam 40a traverses through filter assembly 22 toward a front optics assembly to the eye. Returning from the eye, imaging light beam 42 traverses along the detection axis through a different imaging filter in the filter assembly 22 before arriving at imaging optics assembly 52 followed by imaging sensor 56. At a time delay from the first illumination, a second illumination from illumination assembly 5b, light beam 40b follows a similar light path to collect a retinal image at the opposite inclination angle from the detection axis.

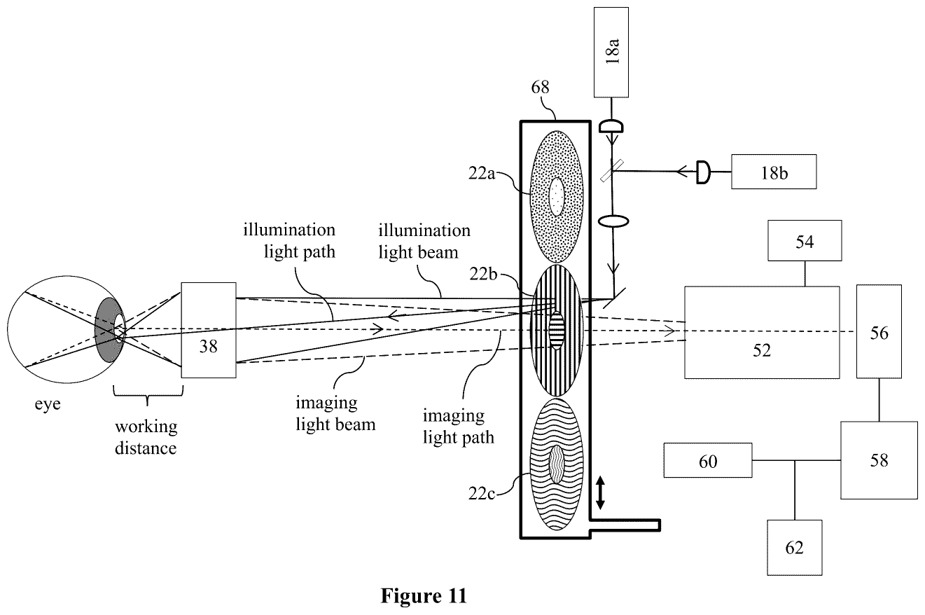

FIG. 10A illustrates an annular cross polarizer filter assembly. For a generic filter assembly 22, an annular cross polarizer filter assembly can have a polarized annular shaped illumination light filter 24 to modify the light in the illumination path and an inner disc-shaped polarized imaging light filter 26 for the purpose of modifying the light in the imaging path. The illumination light filter 24 and imaging light filter 26 can be several different filter pair combinations to serve different application or functional usages. In an embodiment for MSI imaging applications, the annular illumination light filter 24 is a linear polarizer and the inner filter 26 is a similar linear polarizer with its polarization orientation orthogonal to the polarization orientation of the annular polariser. This polarizer filter pair arrangement is used primarily in the present apparatus to suppress residual retro-reflections from the surfaces of objective lens and reduce other non-specific specular reflections or scatters. In one embodiment, to reduce this retroreflection from surfaces of the front optics as well as the anterial elements of the human eye, the polarization orientations of a pair of linear polarizers, are orthogonally aligned. Particularly, an annular outer filter in the illumination light path which is a linear polarizer, is positioned on the outside of an imaging light filter 26 which is a disc shaped linear polarizer with its polarization orientation orthogonal to that of the outer polarizer. Parallel lines indicate the orientation of polarization. The illumination light is launched and passes through the outer linear polarizer which is illumination light filter 24 and becomes linearly polarized in the orientation direction indicated, i.e. only the portion of the illumination light in the same polarisation orientation as the outer polarizer will pass through. Light then goes through the front objective lens and enters the eye through the pupil. The light coming back from the illuminated retina is generally depolarized and it passes through the front objective lens. The portion of the light with the polarization orientation in the same direction of the disc-shaped inner linear polarizer which is imaging light filter 26, whose polarization orientation is orthogonal to that of the outer polarizer or illumination light filter 24, will pass through. The portion of the imaging light and any reflection light with polarization orientation in the orthogonal direction will be rejected. The portion of the imaging light pass through the polarized imaging light filter 26 will be able to propagate further and through the imaging lenses to arrive at the image sensor. The cross-polarizers, specifically illumination light filter 24 and imaging light filter 26, where the filters are polarizers, are preferably coaxially aligned and the polarization of the disk-shaped imaging light polarizer is orientated orthogonally, or at a 90-degree angle, to the ring-shaped or annular polariser, shown as illumination light filter 24. The polarized filter elements can be comprised of varying polarizer types and have different line distances. In one example, a broadband wire grid polarizer pair are utilized to reduce retro reflections from the lens surfaces. wire grid polarizers have a thin layer of aluminum micro-wires attached to a glass substrate or layered between two fused silica windows. Wire grid polarizers can also be lithographically deposited onto glass substrates. The metal wires in wire grid polarizer are typically between about 100 nm to several microns apart.

FIG. 10B is an exploded view of an example filter assembly for use in an apparatus for multi-spectral retinal imaging. Filter assembly 22 has an illumination light filter 24 for receiving incoming light from multi-spectral illumination light sources, and imaging light filter 26 receives light from the retina to filter the light before the light is received by an imaging sensor. Filter frame 70 holds illumination light filter 24 and imaging light filter 26, and filter retainer 72 secures the filters in place. In an embodiment the illumination light filter 24 and imaging light filter 26 are polarization filters that are orthogonally offset in a cross-polarization orientation. Cross-polarization limits illumination light received by the retina to a single polarization, and imaging light received at the imaging sensor is polarized in the orthogonal direction by the imaging light filter 26 to reduce retro reflection. In another embodiment the illumination light filter 24 and imaging light filter 26 is a color filter to reduce the waveband of the illumination light and imaging light. In the case of fundus autofluorescence in particular, the illumination light filter 24 can be at about the excitation wavelength of the retinal pigment to be imaged and the imaging light filter 26 can be around the fluorescence or emission wavelength for detection by the imaging sensor.

FIG. 11 illustrates exchange of filter-pair assemblies in a multi-spectral imaging apparatus. In the present multi-spectral imaging apparatus, filter assemblies can be removed or interchanged and replaced with another filter assembly of different spectral or other optical characteristics. Filter cassette 68 is shown with three filter assemblies 22a, 22b, 22c. For example: assembly 22a may be an amber FAF filter-pair for 660 nm FAF imaging; assembly 22b can be a cross-polarization pair for MSI imaging; assembly 22c can be NIR1 FAF filter-pair for 780 nm FAF. Changing the filter assembly in use can be done by manually or automatically moving the undesired assembly out of the light paths and moving the required assembly into the proper light path position. Means to manually select the desired filter(s) can also be provided. Illumination light from light sources 18a, 18b can be launched through the illumination light filter in filter assembly 22b as shown and directed to objective lens 38, with imaging light returning through the imaging light filter of filter assembly 22b to imaging optics assembly 52 and imaging sensor 56. Also shown are actuator 54, control module 58, display 60, and input device 62.

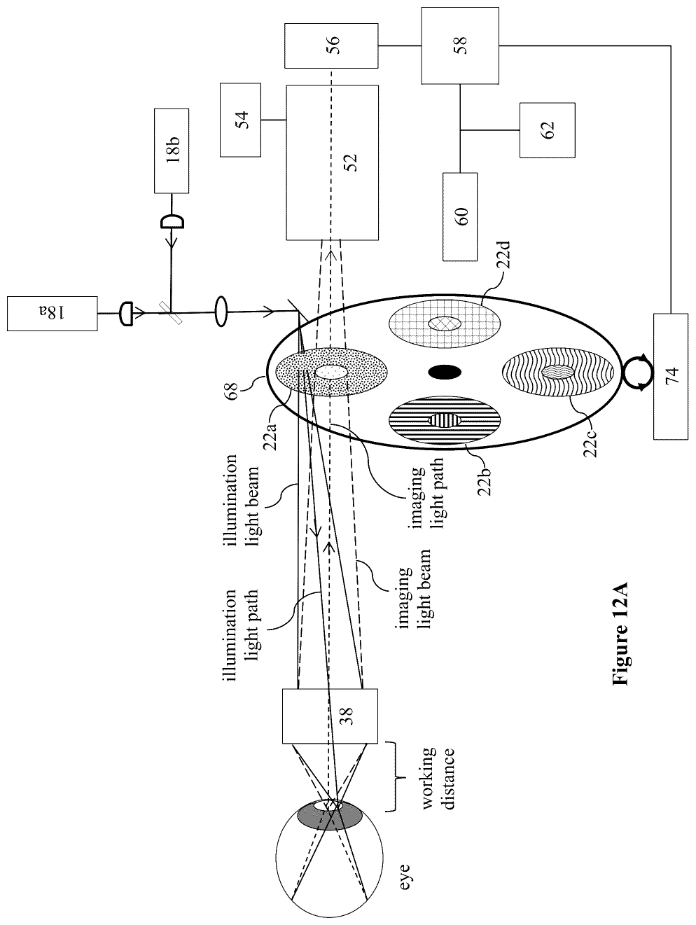

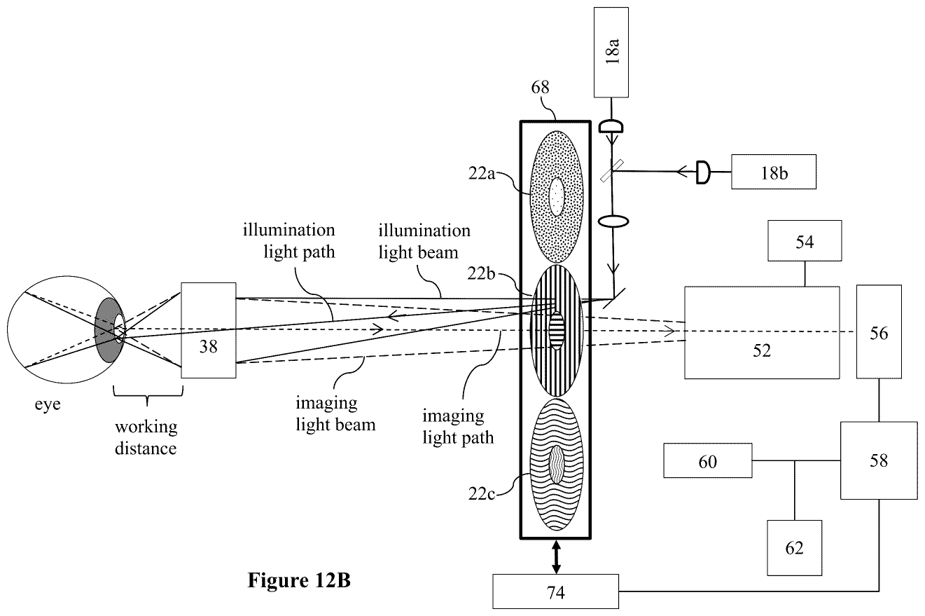

FIG. 12A illustrates an embodiment of an annular filter cassette comprising multiple filter assemblies. Filter cassette 68 provides a mechanism for selecting and interchanging multi filter combination assemblies to provide specific spectral filtering or other optical characteristic filtering such as polarization preference or selection. Filter cassette 68 in an imaging assembly of an apparatus for multi-spectral retinal imaging has filter assemblies 22a, 22b, 22c, 22d. Imaging filter cassette 68 is positioned to enable the selected filter assembly to be in the right position which is perfectly aligned with the illumination light path and the imaging light path. Imaging filter cassette 68 can be controlled by filter assembly actuator 74, such that a particular filter or non-filter in the cassette can be selected. Filter assembly actuator 74 can be controlled by the operator through control module 58. Each filter in the imaging filter cassette 68 can be a polarization filter or a color filter, or a combination of both a polarization filter and a color filter. Each of the filter assemblies 22a, 22b, 22c, 22d can be selected based on the illumination and imaging light desired for imaging. For use in fundus autofluorescence imaging, the excitation light source wavelength for the retinal illumination beam and the corresponding imaging wavelength imaging beam is selected based on the retinal fluorophore(s) being imaged. Illumination light from light sources 18a, 18b can thereby by filtered and sent to objective lens 38 to provide filtered illumination light to the eye. The image capturing by the imaging sensor is synchronized with the illumination source by a trigger from the control module 58. Also shown are imaging optics assembly 52, imaging sensor 56, actuator 54, display 60, and input device 62.

FIG. 12B illustrates an embodiment of a linear filter cassette comprising multiple filter assemblies. The linear filter cassette 68 holds multiple filter assemblies 22a, 22b, 22c which may be one or more polarization filter or color filter. The filter cassette can be connected to and mechanically actuated by a filter assembly actuator 74 which can comprise, for example, a linearly moving mechanism to position the desired filter assembly in the light path. Filter assembly actuator 74 can be controlled by the operator through control module 58. Selection of filters for filtering imaging light before the imaging light enters imaging sensor provides wavelength and/or polarization selection. Illumination light from light sources 18a, 18b can thereby by filtered and sent to objective lens 38 to provide filtered illumination light to the eye. Also shown are imaging optics assembly 52, imaging sensor 56, actuator 54, display 60, and input device 62.

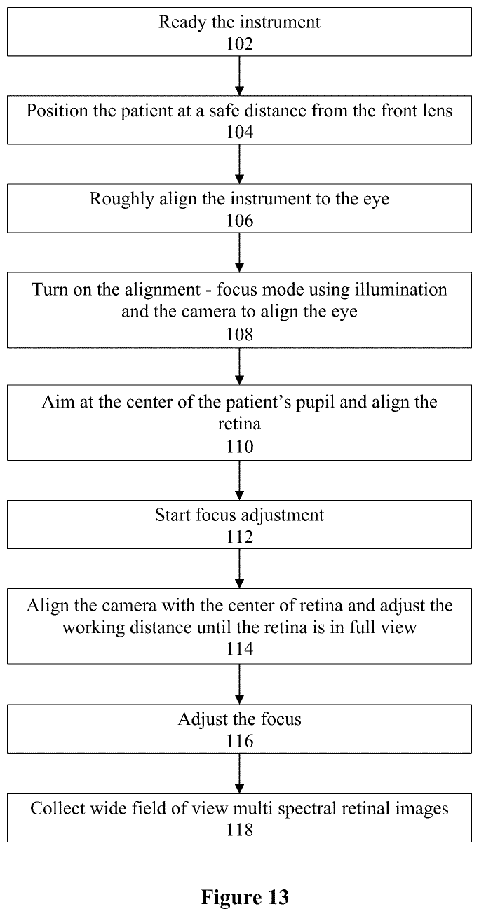

FIG. 13 illustrates a method of obtaining wide field of view retinal imaging including fundus autofluorescence. At first, the instrument is readied 102. The patient is then positioned such that their chin rests securely on a chin rest of the instrument at a distance from the front lens 104, e.g. the eye to be examined is at least 100 mm from the objective lens and the instrument is roughly aligned or visually aligned by the operator to the eye of the patient 106. The instrument alignment/focus mode can be turned on 108. In this mode, an infra-red illumination light, for example at 850 nm, can be turned on and the camera can continuously capture images which can be shown on the display of the instrument. The operator can then move the apparatus to aim at the center of the patient's pupil so that the center of the detection optics is in the center of the eye 110, optionally as displayed on the instrument monitor. Focus adjustment can then start 112 and the apparatus can then be moved towards the patient eye such that the distance between the patient eye and the instrument front objective lens is at a pre-defined working distance. The retina of the eye will be in full plain view on the display screen 114. One method to align and get prepared for retinal imaging with the presently described apparatus for multi-spectral retinal imaging is to use one of the illumination lights intended for retinal imaging. Specifically, by positioning the overall system away from the eye under examination and finding the eye pupil to start the alignment process and then moving the instrument towards the eye by keeping the center of the image sensor aligned as much as possible to the center of the eye. Image display assistance may also be used to indicate the center of the image sensor. A focus or prefocus adjustment of the lens system may also be desirable. Once the imaging system enters full retina view the patient is advised to gaze at a fixation target, which helps to stabilize the eye. The optical system can then be adjusted according to the eye's accommodation for the retina images to be in focus 116 on the image sensor. Focus adjustment is done to enable the best clarity and sharpness of acquired images. This can be realized by manually and/or automatically adjusting the optical components of the present apparatus. In one embodiment, the focus adjustment can be done through motorized actuation mechanism either to move focus adjustment lens or move the image sensor. The retinal imaging system is in focus means the image sensor is at the desired focal plane for retina imaging with respect to the eye's accommodation and images are the sharpest and clearest. This can be achieved by either adjusting the position of the image sensor or a specifically designated focus adjustment lens or optical component. A computational algorithm may also be used to assess the image sharpness and predicts the directional trend, which can generate commands to control the actuator to bring the image to focus. One or more images can then be collected as wide field of view multi spectral retinal images 118. Image capture is based on an image sensor with resolution, performance, and capture rate that match the requirement and quality of the imaging needs. For pulsed illumination, the image sensor is synchronized in capture with the illumination pulsing. Images are captured and subsequently stored electronically on a non-volatile storage medium and may be processed to compensate for various imperfections in the optical system, and to spatially align them. An image sensor with adequate resolution and with high sensitivity and low noise is essential for such implementation. Commercial products such as SONY IMX542 or IMX387 and Complementary Metal-Oxide-Semiconductor (CMOS) sensors can work in the present application. Compound semiconductor sensors, such as GaAsP (Gallium Arsenide Phosphide), GaAs (Gallium Arsenide), or InGaAs (Indium Gallium Arsenide) based image sensors can also be used for longer wavelengths near infrared imaging. Common defects in retinal imaging can then be corrected, such as, for example, unwanted reflections and scatterings, and uneven illuminations. These defects cause degradations of image quality. A number of common techniques can be used to minimise the adverse effect, including but not limited to manufacturing calibration and image processing.

FIG. 14 illustrates a retinal image with wide field of view based on combined retinal images. For a pair of opposed light sources in an illumination assembly, a single image can be taken from an illumination beam from each of the two opposing light sources in an illumination assembly. In particular, a first image taken from a first light source provides a first retinal illumination beam at an inclination angle a relative to the detection axis, and a second image taken from a second and opposing light source provides a second retinal illumination beam at the same inclination angle a relative to the detection axis. In image A, a first retinal image is obtained with a first illumination assembly illuminated, where the light beam is at a first inclination angle relative to the detection axis. Purkinje reflections are shown in A as an overexposed or bright spot on the lower edge of the image, which obscured retinal detail. In image B, a second retinal image is obtained with a second illumination assembly illuminated, where the light beam from the second illumination assembly is coplanar with the light beam from the first illumination assembly and at the same inclination angle from the detection axis but on the opposite side relative to the first illumination beam. The Purkinje reflection, shown as a bright spot, appears on the top edge of the image. This results in the two illumination beams' waists fall on the cornea at different locations, opposite to each other. The fact that Purkinje reflections, i.e. the reflections from cornea and eye lens, appear in the different location, opposite to each other with opposite illumination beam inclinations allows image processing techniques to be applied to each image. More specifically, a slice is taken out the bottom portion of image A which contains the reflection spot, and a similar slice is taken out the top portion of image B that contains the reflection spot. The two images can then be carefully aligned and the two remaining portions of the images can be stitched together to form a near perfect image without the reflection spot as shown in image C.

FIG. 15 illustrates the casing of an apparatus for multi-spectral retinal imaging with wide field of view including multi-spectral fundus auto fluorescence. The presently described retinal imaging apparatus 2 can be housed in any appropriate casing for clinical use.

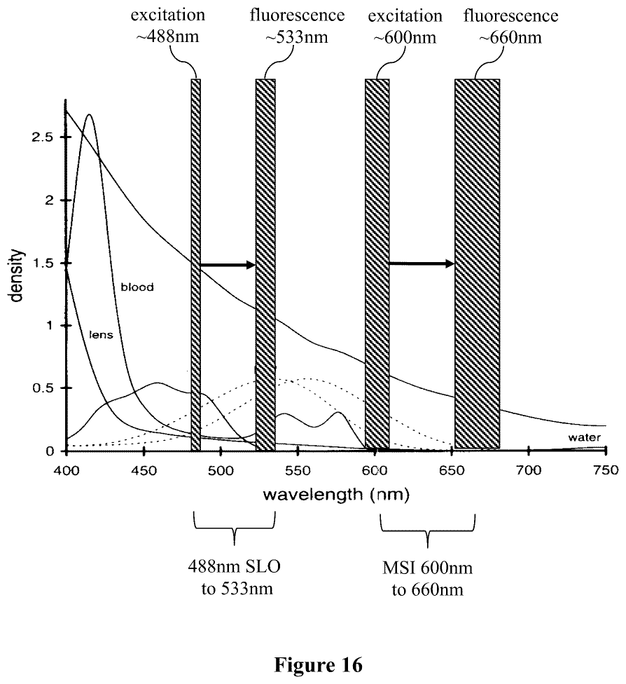

FIG. 16 is an example of the optical spectra for one of the multi-spectral imaging wavelength for autofluorescence (600 nm excitation) in comparison with the commonly used blue autofluorescence by an SLO instrument (488 nm excitation). Autofluorescence (AF) takes advantage of the relationship between the morpho-functional properties of retinal fluorophores and their emission feature, as each fluorophore has a particular AF signature. There is a continuum of the spectral fundus autofluorescence (FAF) from blue to near-infra-red (NIR) because of the combination of the different fluorophores, for example, melanolipofuscin granules of different orders. FAF is interpreted depending on the illumination wavelength used, as different wavelengths will excite different retinal fluorophores. In one example, Kellner et al. (Retina 2010; 30:1:6-15) describe fundus autofluorescence at an excitation wavelength of 488 nm and near infrared autofluorescence at 787 nm to visualize different retinal pigment epithelium alterations in patients with age-related macular degeneration. The fluorescent spectrum can allow for topographical mapping of endogenous retinal fluorophores such as, for example, lipofuscin, melanin, and melanolipofuscin at the level of post-mitotic retinal pigment epithelium and/or photoreceptor (outer segment) complex. The eye naturally has a spectral reflectance, with absorption of light at the fovea, blood, melanin, macular pigment, the lens, and water in the eye. Shown in FIG. 16 over a natural eye reflectance graph from Van de Kraats et al. (The Pathways of Light Measured in Fundus Reflectometry, Vision Research, Volume 36, Issue 15, 1996, Pages 2229-2247) is a graphical representation illustrating long multi-spectral imaging with autofluorescence with short wavelength excitation autofluorescence at 488 nm dominated by retinal pigment epithelium lipofuscin, as well as longer wavelength multi-spectral imaging with autofluorescence at 600 nm excitation. Autofluorescence retinal imaging at 600 nm excitation avoids interaction and mixing with macular pigment. In particular, it has been found that fluorescence emission from retinal pigment epithelium is dominated by oxidized melanin and fluorophores.

Another commonly used FAF technique which may be used with the presently described apparatus is the near-infrared autofluorescence (NIR-AF), which uses an excitation light source at around 780 nm and imaging emission above about 830 nm, beyond the red end of the visible spectrum. The retinal fluorophores that can be examined by NIR-AF are melanin and melanolipofuscin, with a major contribution from retinal pigment epithelium and a smaller contribution from the choroid. Studies have shown that melanin becomes fluorescent at near-infrared wavelengths following oxidization, with an increase of NIR-AF signal in aging patients and at the border of atrophic lesions in age-related macular degeneration (AMD). NIR-AF can be used to detect retinal changes earlier than short wave autofluorescence in patients with geographic atrophy. Therefore, short wave FAF and NIR-FAF have complementary roles in the evaluation of retinal pigments lipofuscin and melanin, and their roles in AMD pathogenesis. Short wavelength FAF, also referred to as blue FAF, can also provide diagnostic analysis of retinal disease states. In particular, FAF can be measured in vivo by spectrophotometry at a broad excitation spectrum that peaks between 490-510 nm. The resulting fluorescence emission is also broad and centered at approximately 600 nm. Other possible excitation wavelengths of interests may be deep Red (660 nm) FAF and NIR2 (810 nm) FAF.

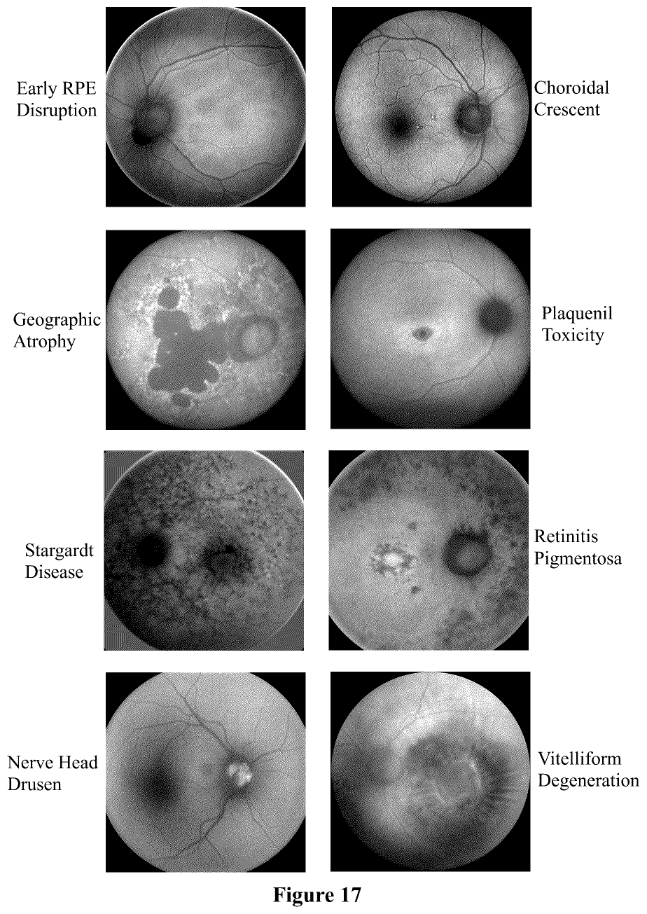

FIG. 17 provides example retinal images for a variety of retinal diseases using multi-spectral imaging. The presently described apparatus can provide FAF imaging at a plurality of different wavelengths, enabling rapid diagnostic application of MSI-FAF to range of diseases of retinitis pigmentosa. Shown are retinal images of early retinal pigment epithelium (RPE) disruption, choroidal crescent, geographic atrophy, Plaquenil toxicity, Stargardt disease, retinitis pigmentosa, nerve head drusen, vitelliform degeneration, which can be imaged and diagnosed using the presently described apparatus. In particular, the use of multi-spectral imaging with FAF at 600 nm excitation with emission at 660 nm provides high contrast imaging of retinal pigment epithelium (RPE). The present method and apparatus can identify areas of the retina dominated by oxidized melanin and fluorophores, with patterns of imaging diagnostic of particular retinal conditions. The disclosed apparatus is particularly effective in identifying RPE atrophy, chronic central serous chorioretinopathy (CSR), retinitis pigmentosa, missing melanin, and is also capable of delineating areas of RPE affected by metabolic stress. Long wavelength FAF imaging, such as 600 nm emission FAF, combined with multi-spectral imaging provide clear and effective diagnostic methods for retinal disease. A hypo-autofluorescence signal can also be observed in conditions with reduced RPE cell number, indicating masking or lower concentration of lipofuscin. RPE atrophy such as in atrophic AMD, fibrosis, presence of intraretinal fluid, and pigment or blood accumulation are all causes for hypo autofluorescence. Hyper autofluorescence signals are often noted in conditions with increased lipofuscin, such as Stargardt's disease, vitelliform macular dystrophy also known as Best disease, and other types of dystrophies.

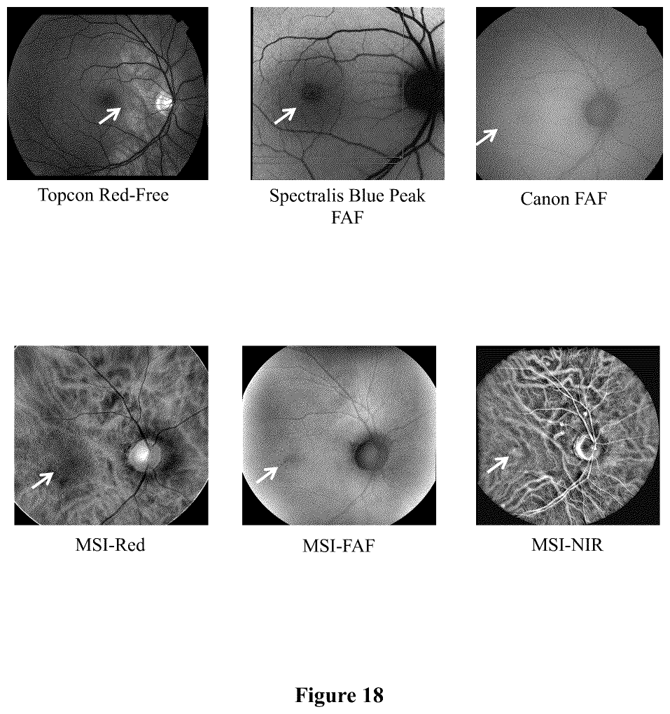

FIG. 18 illustrates a multimodal image case comparing fundus imaging with MSI images and MSI-FAF at 660 nm. Fundus imaging shown on the top row illustrates imaging with Topcon Red-Free, Spectralis Blue Peak FAF, and Canon FAF. MSI-FAF at 660 nm taken with the presently described apparatus are shown on the bottom row. The white arrow in each image is pointing at the same area on the retina as observed with various imaging modalities. Top row images are acquired with red part of spectrum (Topcon-left image), and Fundus Auto fluorescence (center and right image). It is noted that these images showing no observable structural/biomarker or metabolic defects. Bottom row of images has MSI-Red spectrum image (left) showing focal changes in melanin distribution related to health of RPE and early disease progression, MSI-FAF image with mild hypo fluorescent spots indicating correlation with metabolic changes and disease progression as shown in MSI-Red, and MSI-NIR showing deep choriocapillaris defects which is corelated to perfusion and blood supply from choroid. These combined results clearly demonstrate advantage of MSI in finding disease early. As observed, MSI-FAF provides direct observation of early RPE defects not visible with other imaging modalities.

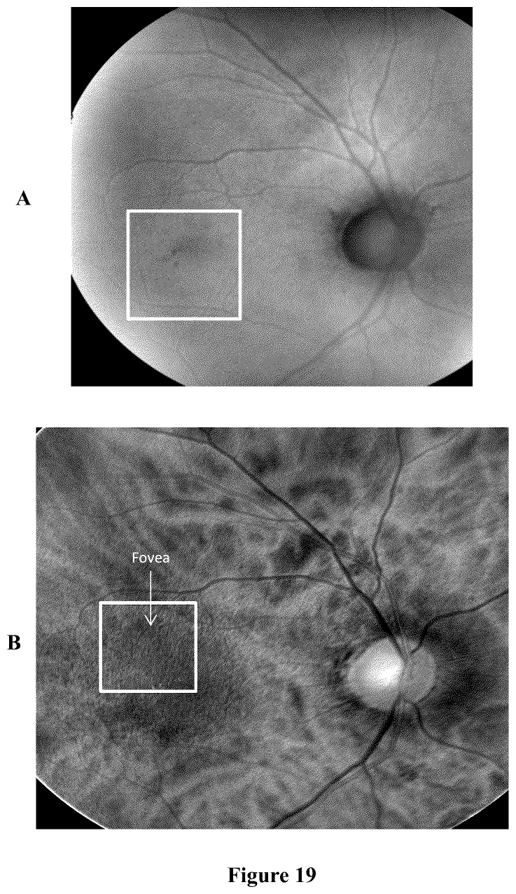

FIG. 19 shows multi-spectral retinal imaging with autofluorescence generated with 600 nm light source. Long FAF is a useful and standard biomarker with high sensitivity to small area of RPE defects. This demonstrates that 600 nm with long FAF is a useful to be a standard biomarker with high sensitivity to small area of RPE defects. Features visible on MSI-600 long FAF results directly from oxidized melanin RPE pigment and has high sensitivity. Shown in image A, MSI-600 long FAF with excitation at 600 nm and auto fluorescent response at and above 660 nm, shows hypo-fluorescent spots at the fovea correlating to absence of melanin and metabolic changes in the RPE indicating early disease signs. Shown in image 1B, MSI-Red image shows small window defects, indicating absence of melanin, correlated to hypo fluorescent spots in image A.

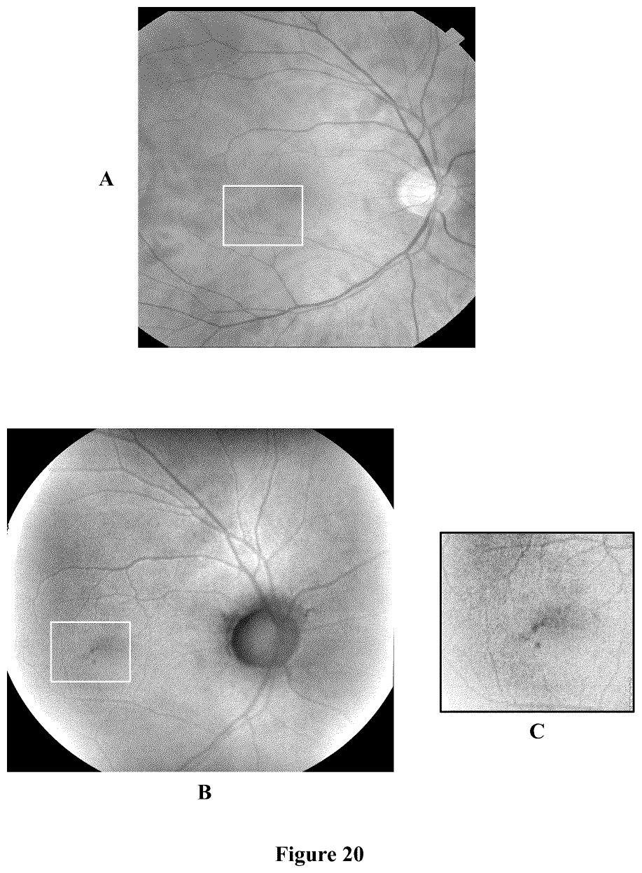

FIG. 20 shows a comparison between a fundus image compared to multi-spectral imaging with fundus autofluorescence at 660 nm in the same eye. Image A shows the red component of the color fundus image using a red pass filter. Image B shows a MSI-FAF image that is result of excitation at MSI-600 nm. Image C shows an enlarged view of the highlighted area in image B with clear dark (hypo fluorescent) spots indicating death of the retinal pigmented epithelium (RPE) tissue.

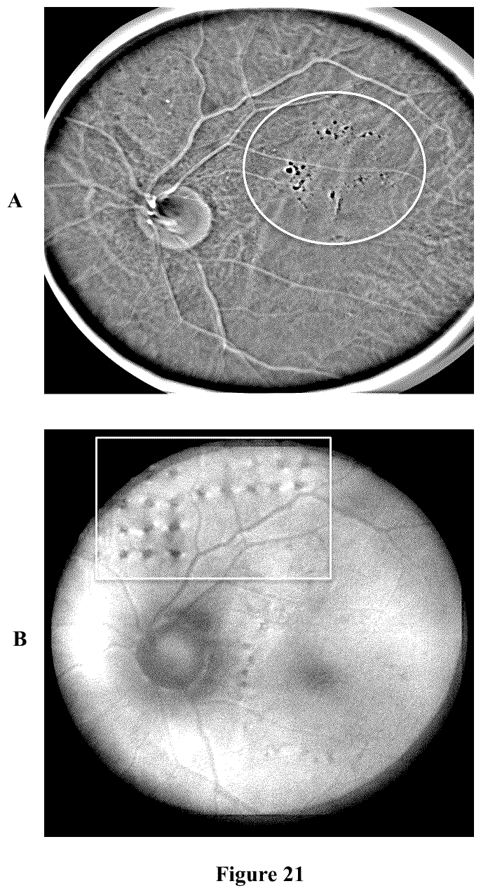

FIG. 21 shows an example of fundus autofluorescence compared to multi-spectral imaging in retinal images in the same eye. Image A shows a retinal image using MSI-NIR, with clear observation of exudates surrounding diabetic macular edema (DME). Within the same eye, image B shows a retinal MSI-FAF image with 600 nm excitation, in superior nasal area shows observable defects due to a grid laser application. The hypo fluorescence (indicating sick retina) and area of hyper fluorescence surrounding laser spots lesions is indicating sick peripheral retina tissue. In addition, in the same image, it is observed that in the central area of the macula a focal area of hypo fluorescence is surrounding an edge of macular edema, indicating direct disease progression. Images generated by a MSI and by a MSI-FAF approach are complementary and provide new information for understanding disease process.

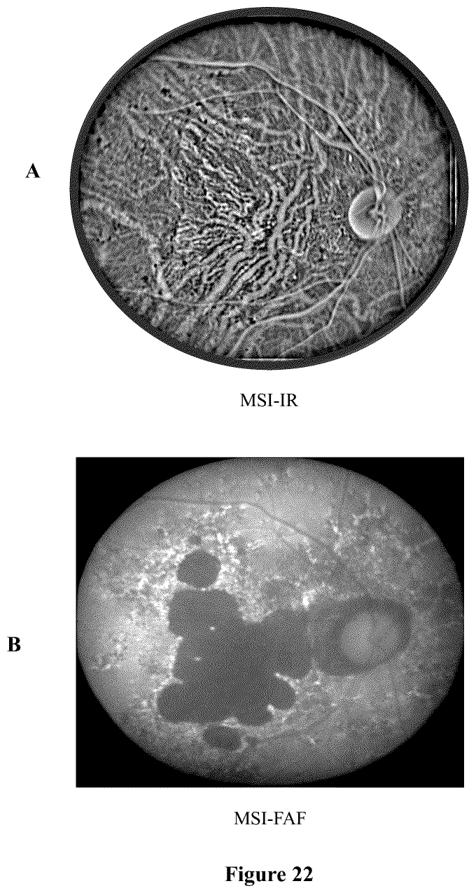

FIG. 22 provides more examples of fundus autofluorescence compared to multi-spectral imaging retinal image in the same eye further deriving benefit of the MSI-FAF metabolic imaging. Image A is a MSI-IR image of the choroid which shows deep vascular network in the choroid and a lack of choriocapillaris vasculature responsible for blood supply to photoreceptors. This is visible in the hypo fluorescent area shown in the image B of the MSI-FAF image indicating that the retina in this area is dead and vision is lost. Surrounding dark areas is hyperfluorescent response of retina indicating where disease is progressing. This can be observed as edge of atrophy in image A.

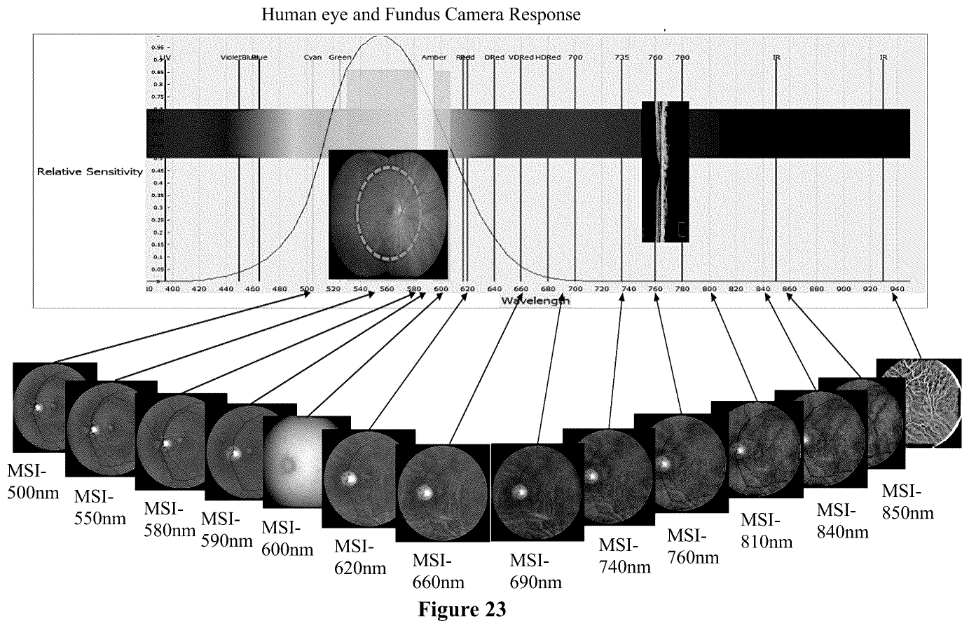

FIG. 23 provides a series of multi-spectral images taken at different wavelengths. Shown are images taken at retinal imaging wavelengths from 475 nm-blue to 900 nm-NIR, to progressively examine the different layers of the retina and choroid. Longer wavelengths penetrate deeper into the structures of the eye and each image provides a different view of the retina depending on wavelength. Each monochromatic spectral slice represents successive images of the fundus as targeted and deliberately selected at different wavelengths. Each wavelength differentially reflects, scatters, and absorbs deeper into the posterior pole, enhancing differential visibility of the retinal and choroidal features. Different illumination light wavelengths can thereby reveal different features within the retina that would be obscured by a white light image. For example, an image taken with an illumination light of 580 nm highlights oxygenated blood vessels and an image taken with an illumination light of 590 nm highlights de-oxygenated blood vessels. These images can also be combined using image processing to provide a clinically valuable map of retinal health. Images can be combined in multiple ways to arrive at a combined optical image with reduced specular reflection or other artifacts. In one example, obtaining a retinal image every 30-50 nm anywhere in the wavelength range of about 475 to about 900 nm provides different information based on the depth of tissue.

All publications, patents and patent applications mentioned in this specification are indicative of the level of skill of those skilled in the art to which this invention pertains and are herein incorporated by reference. The reference to any prior art in this specification is not, and should not be taken as, an acknowledgement or any form of suggestion that such prior art forms part of the common general knowledge.

The invention being thus described, it will be obvious that the same may be varied in many ways. Such variations are not to be regarded as a departure from the scope of the invention, and all such modifications as would be obvious to one skilled in the art are intended to be included within the scope of the following claims.

1. An apparatus for multi-spectral retinal imaging comprising:
a light source assembly comprising a plurality of pairs of opposed illumination assemblies, each illumination assembly comprising:
a first light source configured to emit a first light beam at a first peak wavelength;
a second light source configured to emit a second light beam at a second peak wavelength;
a dichroic mirror to receive light from at least one of the first light source and the second light source and provide an illumination light beam;
a light shaping lens to receive the illumination light beam from the dichroic mirror; and
a directional optical component to direct the illumination light beam at an inclination angle to a detection axis;
a filter assembly comprising an illumination light filter to receive the illumination light beam and an imaging light filter to receive an imaging light beam;
an objective lens to direct the illumination light beam onto a retina; and
an illumination detector on the detection axis to receive projected light from the retina through the imaging light filter,
wherein the inclination angle of the illumination light beam from both of the illumination assemblies in each pair of opposed illumination assemblies is coplanar with the detection axis.

2. The apparatus of claim 1, wherein the first light source and the second light source in each illumination assembly have different peak wavelengths.

3. The apparatus of claim 1, wherein the dichroic mirror has a first side that allows light at the first peak wavelength to be transmitted and a second side that allows light at the second peak wavelength to be reflected.

4. The apparatus of claim 1, wherein the first light beam is orthogonal to the second light beam.

5. The apparatus of claim 1, wherein the illumination light filter is an outer polarization ring at a first polarization and the imaging light beam is an inner polarization disc at a second polarization orthogonal to the first polarization.

6. The apparatus of claim 1, wherein the illumination light filter is an outer color filter ring with a first color filter and the imaging light beam is an inner color filter disc of a second color filter.

7. The apparatus of claim 1, wherein the inclination angle between the illumination light beam and the detection axis is between 2 and 8 degrees.

8. The apparatus of claim 1, further comprising a photosensor to measure illumination intensity of the illumination light beam.

9. The apparatus of claim 1, wherein the filter assembly comprises one or more of polarizing glass, film, broadband metal wire grid polarizer, and aluminum MicroWires.

10. The apparatus of claim 1, wherein the first light source and the second light source comprise one or more of a light emitting diode (LED), LED array, fiber-optic light source, hyper-spectrum laser, wideband tunable laser, and super luminescent diode.

11. The apparatus of claim 1, wherein the imaging sensor is a camera, monochromatic digital image sensor, Complementary Metal-Oxide-Semiconductor (CMOS) sensor, or compound semiconductor sensor.

12. The apparatus of claim 1, wherein the first peak wavelength and the second peak wavelength are between 400 nm to 950 nm.

13. The apparatus of claim 1, further comprising:
a filter cassette comprising one or more filter assembly; and
an actuator to actuate positioning of the filter cassette relative to the detection axis.

14. The apparatus of claim 1, wherein the light source assembly further comprises a fixation target.

15. A method for multi-spectral retinal imaging comprising:
selecting a plurality of imaging wavelengths for retinal imaging; and
acquiring a wide field of view retinal image for each of the selected imaging wavelengths by:
aligning a filter assembly along an imaging detection axis, the filter assembly comprising an outer annular illumination light filter and an inner imaging light filter, the illumination light filter and imaging light filter selected for the selected imaging wavelength;
simultaneously directing light from a first light source through a first dichroic mirror in a first illumination light path at a first inclination angle to the detection axis through the illumination light filter and directing light from a second light source through a second dichroic mirror in a second illumination light path at a second inclination angle to the detection axis through the illumination light filter to illuminate the retina, the first illumination light path and the second illumination light path coplanar with the detection axis; and
receiving imaging light from the retina through the imaging light filter at an imaging sensor along the detection axis to provide a wide field of view retinal image at the selected imaging wavelength.

16. The method of claim 15, wherein the first light source and the second light source are illuminated simultaneously for between 10 and 250 milliseconds.

17. The method of claim 15, wherein the first inclination angle and the second inclination angle to the detection axis is between 2 and 8 degrees.

18. The method of claim 15, wherein the first light source and the second light source have a peak wavelength between 400 nm to 950 nm.

19. The method of claim 15, wherein for each of the plurality of selected imaging wavelengths the first light source and the second light source have the same peak wavelength.

20. The method of claim 15, wherein the illumination light filter and the imaging light filter are cross-polarized or are color filters in different spectral ranges.
